(19) United States
(12) Patent Application Publication
(10) Pub. No.: US 2003/0194732 A1
(43) Pub. Date: Oct. 16, 2003

(54) Interferon-alpha induced gene

(76) Inventors: Jean-Francois Meritet, Paris (FR); Michel Dron, Bourg la Reine (FR); Michael Gerard Tovey, Paris (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/380,558

(22) PCT Filed: Sep. 14, 2001
(86) PCT No.: PCT/GB01/04139

(30) Foreign Application Priority Data
Sep. 14, 2000 (GB) 0022571.4
Oct. 12, 2000 (GB) 0025060

(51) Int. Cl. A61K 38/21; C12Q 1/68; A01K 67/00; C07H 21/04; C12P 21/04

(52) U.S. Cl. 435/6; 424/85.4; 435/69.51; 435/320.1; 435/325; 530/351; 536/23.5; 800/8

(57) ABSTRACT

The present invention relates to identification of a gene upregulated by interferon-α administration corresponding to the cDNA sequences set forth in SEQ. ID. No. 1 or in SEQ. ID. No. 3. Determination of expression products of this gene is proposed as having utility in predicting responsiveness to treatment with interferon-α and other interferons which act at the Type 1 interferon receptor. Therapeutic use of the protein encoded by the same gene is also envisaged.

### FIELD OF THE INVENTION

The present invention relates to identification of a human gene upregulated by interferon-α (IFN-α) administration, the coding sequence of which is believed to be previously unknown. Detection of expression products of this gene may find use in predicting responsiveness to IFN-α and other interferons which act at the Type 1 interferon receptor. Therapeutic use of the isolated novel protein encoded by the same gene is also envisaged.

### BACKGROUND OF THE INVENTION

IFN-α is widely used for the treatment of a number of disorders. Disorders which may be treated using IFN-α include neoplastic diseases such as leukaemia, lymphomas, and solid tumours, AIDS-related Kaposi's sarcoma and viral infections such as chronic hepatitis. IFN-α has also been proposed for administration via the oromucosal route for the treatment of autoimmune, mycobacterial, neurodegenerative, parasitic and viral disease. In particular, IFN-α has been proposed, for example, for the treatment of multiple sclerosis, leprosy, tuberculosis, encephalitis, malaria, cervical cancer, genital herpes, hepatitis B and C, HIV, HPV and HSV-1 and 2. It has also been suggested for the treatment of arthritis, lupus and diabetes. Neoplastic diseases such as multiple myeloma, hairy cell leukaemia, chronic myelogenous leukaemia, low grade lymphoma, cutaneous T-cell lymphoma, carcinoid tumours, cervical cancer, sarcomas including Kaposi's sarcoma, kidney tumours, carcinomas including renal cell carcinoma, hepatic cellular carcinoma, nasopharyngeal carcinoma, haematological malignancies, colorectal cancer, glioblastoma, laryngeal papillomas, lung cancer, colon cancer, malignant melanoma and brain tumours are also suggested as being treatable by administration of IFN-α via the oromucosal route, i.e. the oral route or the nasal route.

IFN-α is a member of the Type 1 interferon family, which exert their characteristic biological activities through interaction with the Type 1 interferon receptor. Other Type 1 interferons include IFN-β, IFN-ω and IEN-τ.

Unfortunately, not all potential patients for treatment with a Type 1 interferon such as interferon-α, particularly, for example, patients suffering from chronic viral hepatitis, neoplastic disease and relapsing remitting multiple sclerosis, respond favourably to Type 1 interferon therapy and only a fraction of those who do respond exhibit long-term benefit. The inability of the physician to confidently predict the therapeutic outcome of Type 1 interferon treatment raises serious concerns as to the cost-benefit ratio of such treatment, not only in terms of wastage of an expensive biopharmaceutical and lost time in therapy, but also in terms of the serious side effects to which the patient is exposed. Furthermore, abnormal production of IFN-α has been shown to be associated with a number of autoimmune diseases. For these reasons, there is much interest in identifying Type 1 interferon responsive genes since Type1 interferons exert their therapeutic action by modulating the expression of a number of genes. Indeed, it is the specific pattern of gene expression induced by Type 1 interferon treatment that determines whether a patient will respond favourably or not to the treatment.

### SUMMARY OF THE INVENTION

A human gene cDNA has now been identified as corresponding to a mouse gene upregulated by administration of IFN-α by an oromucosal route or intravenously and is believed to represent a novel DNA. The corresponding human gene is thus now also designated an IFN-α upregulated gene.

The protein encoded by the same gene is referred to below as HUIFRG46/ADIR (ATP dependent IFN responsive) protein. This protein, and functional variants thereof, are now envisaged as therapeutic agents, in particular for use as an anti-viral, anti-tumour or immunomodulatory agent. For example, they may be used in the treatment of autoimmune, mycobacterial, neurodegenerative, parasitic or viral disease, arthritis, diabetes, lupus, multiple sclerosis, leprosy, tuberculosis, encephalitis, malaria, cervical cancer, genital herpes, hepatitis B or C, HIV, HPV, HSV-1 or 2, or neoplastic disease such as multiple myeloma, hairy cell leukaemia, chronic myelogenous leukaemia, low grade lymphoma, cutaneous T-cell lymphoma, carcinoid tumours, cervical cancer, sarcomas including Kaposi's sarcoma, kidney tumours, carcinomas including renal cell carcinoma, hepatic cellular carcinoma, nasopharyngeal carcinoma, haematological malignancies, colorectal cancer, glioblastoma, laryngeal papillomas, lung cancer, colon cancer, malignant melanoma or brain tumours. In other words, such a protein may find use in treating any Type 1 interferon treatable disease.

Determination of the level of HuIFRG46/ADIR protein or a naturally-occurring variant thereof, or the corresponding mRNA, in cell samples of Type 1 interferon-treated patients, e.g. patients treated with IFN-α, e.g. such as by the oromucosal route or intravenously, may also be used to predict responsiveness to such treatment. It has additionally been found that alternatively, and more preferably, such responsiveness may be judged, for example, by treating a sample of human peripheral blood mononuclear cells in vitro with a Type 1 interferon and looking for upregulation or downregulation of an expression product, preferably mRNA, corresponding to the HuIFRG46/ADIR gene.

According to a first aspect of the invention, there is thus provided an isolated polypeptide comprising;

(i) the amino acid sequence of SEQ ID NO: 2 or of SEQ ID NO: 4;

(ii) a variant thereof having substantially similar function, e.g. an immunomodulatory activity and/or an anti-viral activity and/or an anti-tumour activity; or

(iii) a fragment of (i) or (ii) which retains substantially similar function, e.g. an immunomodulatory activity and/or an anti-viral activity and/or an anti-tumour activity.

The invention also provides such a protein for use in therapeutic treatment of a human or non-human animal, more particularly for use as an antiviral, anti-tumour or immunomodulatory agent. As indicated above, such use may extend to any Type 1 interferon treatable disease. Such a protein may be used in combination with an anti cancer drug for the therapy of cancer.

According to another aspect of the invention, there is provided an isolated polynucleotide encoding a polypeptide of the invention as defined above or a complement thereof. Such a polynucleotide will typically include a sequence comprising:

(a) the nucleic acid of SEQ. ID. No. 1 or of SEQ. ID. NO. 3 or the coding sequence thereof and/or a sequence complementary thereto;

(b) a sequence which hybridises, e.g. under stringent conditions, to a sequence complementary to a sequence as defined in (a);

(c) a sequence which is degenerate as a result of the genetic code to a sequence as defined in (a) or (b);

(d) a sequence having at least 60% identity to a sequence as defined in (a), (b) or (c).

The invention also provides;

an expression vector which comprises a polynucleotide of the invention and which is capable of expressing a polypeptide of the invention;

a host cell containing an expression vector of the invention;

an antibody or a fragment thereof which retains antigen-binding capability specific for a polypeptide of the invention;

a method of treating a subject having a Type 1 interferon treatable disease, a viral disease or of treating or preventing cancer in a patient, which method comprises administering to the said patient an effective amount of HuIFRG46/ADIR protein or a functional variant thereof

use of such a polypeptide in the manufacture of a medicament for use in therapy as an anti-viral or anti-tumour or immunomodulatory agent, more particularly for use in treatment of a Type I interferon treatable disease;

use of such a polypeptide or polynucleotide in cancer therapy and use of such a polypeptide or polynucleotide in combination with an anticancer drug or treatment as a combined preparation for simultaneous, separate or sequential use in cancer therapy;

a pharmaceutical composition comprising a polypeptide of the invention and a pharmaceutically acceptable carrier or diluent;

a product containing a polypeptide or polynucleotide of the invention and an anti-cancer drug, which product is suitable for use as a combined preparation for simultaneous, separate or sequential use in cancer therapy;

a method of producing a polypeptide of the invention, which method comprises maintaining host cells of the invention under conditions suitable for obtaining expression of the polypeptide and isolating the said polypeptide;

a pharmaceutical composition comprising such a polynucleotide and a pharmaceutically acceptable carrier or diluent;

a method of treating a subject having a Type 1 interferon treatable disease, or a viral disease, or of treating or preventing cancer in a patient, which method comprises administering to said patient an effective amount of such a polynucleotide;

use of such a polynucleotide in the manufacture of a medicament, e.g. a vector preparation, for use in therapy as an anti-viral, anti-tumour or immunomodulatory agent, more particularly for use in treating a Type 1 interferon treatable disease; and

a method of identifying a compound having immunomodulatory activity and/or anti-viral activity and/or anti-tumour activity comprising providing a cell capable of expressing HuIFRG46/ADIR protein or a naturally occurring variant thereof, incubating said cell with a compound under test and monitoring for upregulation of HuIFRG46/ADIR gene expression.

In a further aspect, the invention provides a set of primers which sequences within the polynucleotides of the invention, particularly the sequences of SEQ ID Nos 1 or 3 or the complements thereof. The invention also provides nucleic acid probes derived from polynucleotides of the invention which are suitable for the selective detection of the sequences of SEQ ID Nos 1 or 3 or the complements thereof.

In a still further aspect, the invention provides a method of predicting responsiveness of a patient to treatment with a Type 1 interferon, e.g. IFN-α treatment (such as IFN-α treatment by the oromucosal route or a parenteral route, for example, intravenously, subcutaneously, or intramuscularly), which comprises determining the level of HuIFRG46/ADIR protein or a naturally-occurring variant thereof, e:g. an allelic variant, or the corresponding mRNA, in a cell sample from said patient, e.g. a blood sample, wherein said sample is obtained from said patient following administration of a Type 1 interferon, e.g. IFN-α by an oromucosal route or intravenously, or is treated prior to said determining with a Type 1 interferon such as IFN-α in vitro. The invention also extends to kits for carrying out such testing.

### BRIEF DESCRIPTION OF THE FIGURES

FIG. 1: cDNA clones isolated from a BALB/C mouse spleen cDNA library were sequenced and assembled. The longest ORF is represented as the shaded area and the sequence of the band isolated by differential display is emboldened. Potential polyadenylation sites are underlined.

FIG. 2: The amino acid sequence deduced from the mouse ORF was compared to the sequence of the human ADIR protein using the MULTALIN alignment program. Identities are boxed in black and similarities in grey. The typical motifs of ATP binding proteins, A,−B, and Box IV are indicated. Only the putative sites for phosphorylation (PKC, protein kinase C, CK2, casein kinase II; TK, tyrosine kinase) and for glycosylation (Gly) conserved in both human and mouse sequences are indicated.

FIG. 3: Characterisation and tissue distribution of human HuIFRG46/ADIR transcripts. 1 to 14: Brain (whole brain, amygdala, caudate nucleus, cerebellum, cerebral cortex, frontal lobe, hippocampus, medulla oblongata. occipital lobe, putamen, substantia nigra, temporal lobe, thalamus, subthalamic nucleus) 15 to 20 spinal cord. heart, aorta, skeletal muscle, colon, bladder. 21 to 30 uterus, prostate, stomach, testis, ovary, pancreas, pituitary gland, adrenal gland. thyroid gland, salivary gland. 31 to 43 mammary gland, kidney, liver, small intestine, spleen, thymus, peripheral leukocyte, lymph node, bone marrow. appendix, lung, trachea, placenta. 44 to 50 fetal tissues (brain, heart, kidney, liver, spleen, thymus, lung.) Most of the tissues are pools of several individuals except for the cerebral cortex and adult liver. The fetal mRNA are pools from at least 14 embryos of ages comprised between 17 to 25 weeks. The mRNA samples dotted on the membrane have been normalized to the mRNA expression level of eight different housekeeping genes.

FIG. 4: Organization of the human ADIR gene. Exons on human chromosome 1 are represented as gray boxes and prominent protein motifs encoded by each exon are indicated (A). Alternative splicing using exon 7 would generate a protein termed ADIR2 with a modified C terminus and devoid of the Box IV motif present in ADIR1 (B).

FIG. 5: Homology of ADIR with the Torsin and the Clp/HSP100 families of proteins. Alignment of the human ADIR, human and mouse Torsin A and Torsin B proteins, and Torsin-like sequences from the fruit-fly Drosophila Melanogaster and from the nematode C. elegans (A). Dark shading indicates identities, and light shading indicates similarities. Conservation is shown in the consensus sequence; identical amino acids are represented by uppercase emboldened letters and similar amino acid by uppercase or lowercase letters. Highly conserved sequences with potential functional implications are boxed in the consensus sequence. Arrows indicate the position of conserved cysteines. (B). Alignment of ADIR, Triticum aestivum heat shock protein 101, a member the HSPIO1/Clp family of proteins, and mouse SKD3

FIG. 6: Effect of HuIFRG46/ADIR on cell proliferation in the presence of 5 μM 5-FU. Results are expressed as the mean and standard error of six separate cultures per group for each time point.

FIG. 7: Effect of HuIFRG46/ADIR on cell proliferation

FIG. 8: Effect of HuIFRG46/ADIR on the replication of Vesicular Stomatitis Virus

FIG. 9; Effect of HuIFRG46/ADIR on the antitumor activity of 5-FU on the growth of human tumors in nude mice.

### BRIEF DESCRIPTION OF THE SEQUENCES

SEQ. ID. No.1 is the amino acid sequence of human protein HuIFRG46/ADIR and its encoding cDNA.

SEQ. ID. No.2 is the amino acid sequence alone of HuIFRG46/ADIR protein.

SEQ. ID. No.3 is the amino acid sequence of a variant of human protein HuIFRG46/ADIR and its encoding cDNA.

SEQ. ID. No.4 is the amino acid sequence alone of a variant of HuIFRG46/ADIR protein.

SEQ ID No.5 is the DNA sequence of the mouse HuIFRG46/ADIR cDNA.

SEQ ID No.6 is the amino acid sequence of the mouse HuIFRG46/ADIR protein.

SEQ ID No.7 is the DNA sequence of the band isolated by differential display.

SEQ ID No.8 is the DNA sequence of the ISRE identified in the mouse.

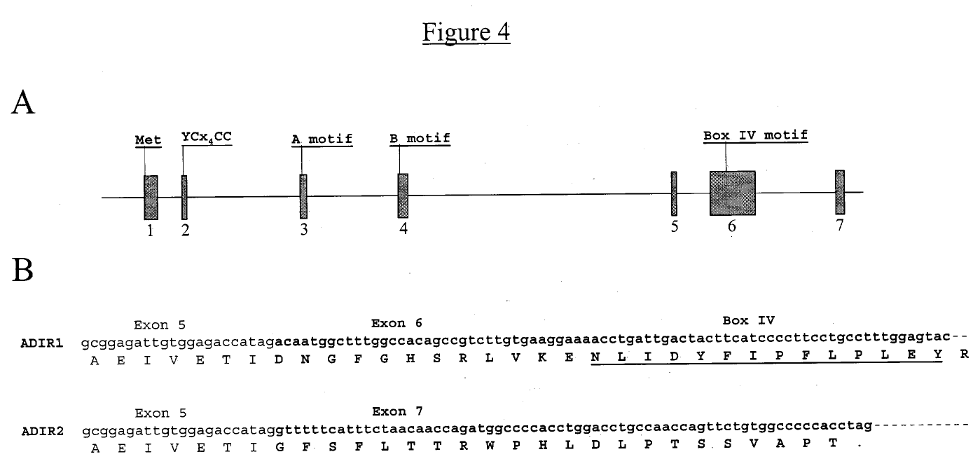

SEQ ID Nos. 9 to 12 indicate the effects of alternative splicing using exon 7 on HuIFRG46/ADIR1 and HuIFRG46/ADIR2 (see FIG. 4).

SEQ ID Nos 13 to 19 are the result of alignment of the following proteins: SEQ ID No.13, HuIFRG46/ADIR; SEQ ID No.14, human Torsin A; SEQ ID No.15, mouse Torsin A; SEQ ID No.16, human Torsin B; SEQ ID No.17, mouse Torsin B; SEQ ID No.18, Drosophila melanogaster Torsin-like protein; SEQ ID Nos 19 to 21, C. elegans Torsin like proteins Y37A1B.13, YUY1 and Y37A1B.12 respectively.

SEQ ID Nos 22 to 24 are the results of the alignment of the following proteins: SEQ ID No.22, HuIFRG46/ADIR; SEQ ID N6.23, Triticum aestivum heat shock protein 101; SEQ ID No.24, mouse SKD3.

### DETAILED DESCRIPTION OF THE INVENTION

As indicated above, human protein HuIFRG46/ADIR and functional variants thereof are now envisaged as therapeutically useful agents, more particularly for use as an anti-viral, anti-tumour or immunomodulatory agent.

A variant of HuIFRG46/ADIR protein for this purpose may be a naturally occurring variant, either an allelic variant or species variant, which has substantially the same functional activity as HuIFRG46/ADIR protein and is also upregulated in response to administration of IFN-α. A variant may be ATP dependent. Alternatively, a variant of HuIFRG46/ADIR protein for therapeutic use may comprise a sequence which varies from SEQ. ID. No. 2 or from SEQ. ID. No. 4 but which is a non-natural mutant.

The term “functional variant” refers to a polypeptide which has the same essential character or basic function of HuIFRG46/ADIR protein. The essential character of HuIFRG46/ADIR protein may be deemed to be anti-viral activity and/or anti-tumour activity. A functional variant polypeptide may act additionally or alternatively as an immunomodulatory peptide

A functional variant for use in the treatment of cancer may be any variant which retains the ability to inhibit cell proliferation or stimulate apoptosis, either alone, or in combination with an anticancer drug. Desired anticancer activity may, for example, be tested using methods as described in Examples 7 and 9.

Desired anti-viral activity may, for example, be tested as follows: A sequence encoding a variant to be tested is cloned into a retroviral vector such as a retroviral vector derived from the Moloney murine leukaemia virus (MoMuLV) containing the viral packaging signal ψ, and a drug-resistance marker. A pantropic packaging cell line containing the viral gag, and pol, genes is then co-transfected with the recombinant retroviral vector and a plasmid, pVSV-G, containing the vesicular stomatitis virus envelope glycoprotein in order to produce high-titre infectious replication incompetent virus (Burns et al., Proc. Natl. Acad. Sci. USA 84, 5232-5236). The infectious recombinant virus is then used to transfect interferon sensitive fibroblasts or lymphoblastoid cells and cell lines that stably express the variant protein are then selected and tested for resistance to virus infection in a standard interferon bio-assay (Tovey et al., Nature, 271, 622-625, 1978). Growth inhibition using a standard proliferation assay (Mosmann, T., J. Immunol. Methods, 65, 55-63, 1983) and expression of MHC class I and class II antigens using standard techniques may also be determined. Antiviral activity may be tested by following the methods laid out in Example 8.

A desired functional variant of HuIFRG46/ADIR may consist essentially of the sequence of SEQ. ID. No. 2 or of SEQ. ID. No. 4. A functional variant of SEQ. ID. No.2 or of SEQ. ID. No. 4 may be a polypeptide which has a least 60% to 70% identity, preferably at least 80% or at least 90% and particularly preferably at least 95%, at least 97% or at least 99% identity with the amino acid sequence of SEQ. ID. No. 2 or of SEQ. ID. No. 4 over a region of at least 20, preferably at least 30, for instance at least 100 contiguous amino acids or over the full length of SEQ. ID. No. 2 or of SEQ. ID. No. 4. Methods of measuring protein identity are well known in the art.

Amino acid substitutions may be made, for example from 1, 2 or 3 to 10, 20 or 30 substitutions. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Variant polypeptide sequences for therapeutic use in accordance with the invention may be shorter polypeptide sequences, for example, a peptide of at least 20 amino acids or up to 50, 60, 70, 80, 100, 150 or 200 amino acids in length is considered to fall within the scope of the invention provided it retains appropriate biological activity of HuIFRG46/ADIR protein. In particular, but not exclusively, this aspect of the invention encompasses the situation when the variant is a fragment of a complete natural naturally-occurring protein sequence.

Also encompassed by the invention are modified forms of HuIFRG46/ADIR protein and fragments thereof which can be used to raise anti-HuIFRG46/ADIR protein antibodies. Such variants will comprise an epitope of the HuIFRG46/ADIR protein.

Polypeptides of the invention may be chemically modified, e.g. post-translationally modified. For example, they may be glycosylated and/or comprise modified amino acid residues. They may, also be modified by the addition of a sequence at the N-terminus and/or C-terminus, for example by provision of histidine residues or a T7 tag to assist their purification or by the addition of a signal sequence to promote insertion into the cell membrane. Such modified polypeptides fall within the scope of the term “polypeptide” of the invention.

A polypeptide of the invention may be labelled with a revealing label. The revealing label may be any suitable label which allows the polypeptide to be detected. Suitable labels include radioisotopes such as ¹²⁵I, ³⁵S or enzymes, antibodies, polynucleotides and linkers such as biotin. Labelled polypeptides of the invention may be used in assays. In such assays it may be preferred to provide the polypeptide attached to a solid support. The present invention also relates to such labelled and/or immobilised polypeptides packaged in the form of a kit in a container. The kit may optionally contain other suitable reagent(s), control(s) or instructions and the like.

The polypeptides of the invention may be made synthetically or by recombinant means. Such polypeptides of the invention may be modified to include non-naturally occurring amino acids, e.g. D amino acids. Variant polypeptides of the invention may have modifications to increase stability in vitro and/or in vivo. When the polypeptides are produced by synthetic means, such modifications may be introduced during production. The polypeptides may also be modified following either synthetic or recombinant production.

A number of side chain modifications are known in the protein modification art and may be present in polypeptides of the invention. Such modifications include, for example, modifications of amino acids by reductive alkylation by reaction with an aldehyde followed by reduction with NaBH₄, amidination with methylacetimidate or acylation with acetic anhydride.

Polypeptides of the invention will be in substantially isolated form. It will be understood that the polypeptides may be mixed with carriers or diluents which will not interfere with the intended purpose of the polypeptide and still be regarded as substantially isolated. A polypeptide of the invention may also be in substantially purified form, in which case it will generally comprise the polypeptide in a preparation in which more than 90%, for example more than 95%, 98% or 99%, by weight of polypeptide in the preparation is a polypeptide of the invention.

Polynucleotides

The invention also includes isolated nucleotide sequences that encode HuIFRG46/ADIR protein or a variant thereof as well as isolated nucleotide sequences which are complementary thereto. The nucleotide sequence may be DNA or RNA, single or double stranded, including genomic DNA, synthetic DNA or cDNA. Preferably the nucleotide sequence is a DNA sequence and most preferably, a cDNA sequence.

As indicated above, such a polynucleotide will typically include a sequence comprising:

(a) the nucleic acid of SEQ. ID. No. 1 or of SEQ. ID. No. 3 or the coding sequence thereof and/or a sequence complementary thereto;

(b) a sequence which hybridises, e.g. under stringent conditions, to a sequence complementary to a sequence as defined in (a);

(c) a sequence which is degenerate as a result of the genetic code to a sequence as defined in (a) or (b);

(d) a sequence having at least 60% identity to a sequence as defined in (a), (b) or (c).

Polynucleotides comprising an appropriate coding sequence can be isolated from human cells or synthesised according to methods well known in the art, as described by way of example in Sambrook et al. (1989) Molecular Cloning: A Laboratory Manual, 2^{nd} edition, Cold Spring Harbor Laboratory Press.

Polynucleotides of the invention may include within them synthetic or modified nucleotides. A number of different types of modification to polynucleotides are known in the art. These include methylphosphonate and phosphothioate backbones, addition of acridine or polylysine chains at the 3′ and/or 5′ ends of the molecule. Such modifications may be carried out in order to enhance the in vivo activity or lifespan of polynucleotides of the invention.

Typically a polynucleotide of the invention will include a sequence of nucleotides, which may preferably be a contiguous sequence of nucleotides, which is capable of hybridising under selective conditions to the coding sequence or the complement of the coding sequence of SEQ. ID. No. 1 or of SEQ. ID. No. 3. Such hybridisation will occur at a level significantly above background. Background hybridisation may occur, for example, because of other cDNAs present in a cDNA library. The signal level generated by the interaction between a polynucleotide of the invention and the coding sequence or complement of the coding sequence of SEQ. ID. No. 1 or SEQ. ID. No. 3 will typically be at least 10 fold, preferably at least 100 fold, as intense as interactions between other polynucleotides and the coding sequence of SEQ. ID. No. 1 or of SEQ. ID. No. 3. The intensity of interaction may be measured, for example, by radiolabelling the probe, e.g. with ³²p. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.03M sodium citrate at about 60° C.).

The coding sequence of SEQ ID No: 1 or of SEQ. ID. No. 3 may be modified by nucleotide substitutions, for example from 1, 2 or 3 to 10, 25, 50 or 100 substitutions. Degenerate substitutions may be made and/or substitutions may be made which would result in a conservative amino acid substitution when the modified sequence is translated, for example as shown in the table above. The coding sequence of SEQ. ID. NO: 1 or of SEQ. ID. No. 3 may alternatively or additionally be modified by one or more insertions and/or deletions and/or by an extension at either or both ends.

A polynucleotide of the invention capable of selectively hybridising to a DNA sequence selected from SEQ. ID No.1 or from SEQ. ID. No.3, the coding sequence thereof and DNA sequences complementary thereto will be generally at least 70%, preferably at least 80 or 90% and more preferably at least 95% or 97%, homologous to the target sequence. This homology may typically be over a region of at least 20, preferably at least 30, for instance at least 40, 60 or 100 or more contiguous nucleotides.

Any combination of the above mentioned degrees of homology and minimum sizes may be used to define polynucleotides of the invention, with the more stringent combinations (i.e. higher homology over longer lengths) being preferred. Thus for example a polynucleotide which is at least 80% homologous over 25, preferably over 30 nucleotides forms may be found suitable, as may be a polynucleotide which is at least 90% homologous over 40 nucleotides.

A variant polynucleotide according to the present invention may include the putative ISRE sequence of SEQ ID No.8.

Homologues of polynucleotide or protein sequences as referred to herein may be determined in accordance with well-known means of homology calculation, e.g. protein homology may be calculated on the basis of amino acid identity (sometimes referred to as “hard homology”). For example the UWGCG Package provides the BESTFIT program which can be used to calculate homology, for example used on its default settings, (Devereux et al. (1984) Nucleic Acids Research 12, 387-395). The PILEUP and BLAST algorithms can be used to calculate homology or line up sequences or to identify equivalent or corresponding sequences, typically used on their default settings, for example as described in Altschul S. F. (1993) J. Mol. Evol. 36,290-300; Altschul, S. F. et al. (1990) J. Mol. Biol. 215,403-10.

Software for performing BLAST analyses is publicly available through the National Center for Biotechnology Information (http://www.ncbi.nln.nih.gov/). This algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89,10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Polynucleotides according to the invention have utility in production of the proteins according to the invention, which may take place in vitro, in vivo or ex vivo. In such a polynucleotide, the coding sequence for the desired protein of the invention will be operably-linked to a promoter sequence which is capable of directing expression of the desired protein in the chosen host cell. Such a polynucleotide will generally be in the form of an expression vector. Polynucleotides of the invention, e.g. in the form of an expression vector, which direct expression in vivo of a polypeptide of the invention having immunomodulatory activity and/or anti-viral activity and/or anti-tumour activity may also be used as a therapeutic agent.

Expression vectors for such purposes may be constructed in accordance with conventional practices in the art of recombinant DNA technology. They may, for example, involve the use of plasmid DNA. They may be provided with an origin of replication. Such a vector may contain one or more selectable markers genes, for example an ampicillin resistance gene in the case of a bacterial plasmid. Other features of vectors of the invention may include appropriate initiators, enhancers and other elements, such as for example polyadenylation signals which may be desirable, and which are positioned in the correct orientation, in order to allow for protein expression. Other suitable non-plasmid vectors would be apparent to persons skilled in the art. By way of further example in this regard reference is made again to Sambrook et al., 1989 (supra). Such vectors additionally include, for example, viral vectors. Examples of suitable viral vectors include herpes simplex viral vectors, replication-defective retroviruses, including lentiviruses, adenoviruses, adeno-associated virus, HPV viruses (such as HPV-16 and HPV-18) and attenuated influenza virus vectors.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium and β-actin promoters. Viral promoters such as the SV40 large T antigen promoter or adenovirus promoters may also be used. Other examples of viral promoters which may be employed include the Moloney munne leukaemia virus long terminal repeat (MMLV LTR), the rous sarcoma virus (RSV) LTR promoter, the human cytomegalovirus (CMV) IE promoter, and HPV promoters, particularly the HPV upstream regulatory region (URR). Other suitable promoters will be well-known to those skilled in the recombinant DNA art.

An expression vector of the invention may further include sequences flanking the coding sequence for the desired polypeptide of the invention providing sequences homologous to eukaryotic genomic sequences, preferably mammalian genomic sequences, or viral genomic sequences. This will allow the introduction of such polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell.

The invention also includes cells in vitro, for example prokaryotic or eukaryotic cells, which have been modified to express the HuIFRG46/ADIR protein or a variant thereof. Such cells include stable, e.g. eukaryotic, cell lines wherein a polynucleotide encoding HuIFRG46/ADIR protein or a variant thereof is incorporated into the host genome. Host cells of the invention may be mammalian cells or insect cells, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably a cell line may be chosen which is not only stable, but also allows for mature glycosylation of a polypeptide. Expression may, for example, be achieved in transformed oocytes.

A polypeptide of the invention may be expressed in cells of a transgenic non-human animal, preferably a mouse. A transgenic non-human animal capable of expressing a polypeptide of the invention is included within the scope of the invention.

Polynucleotides according to the invention may also be inserted into vectors as described above in an antisense orientation in order to provide for the production of antisense sequences. Antisense RNA or other antisense polynucleotides may also be produced by synthetic means.

A polynucleotide, e.g. in the form of an expression vector, capable of expressing in vivo an antisense sequence to a coding sequence for the amino acid sequence defined by SEQ. ID. No. 2 or by SEQ. ID. No. 4, or a naturally-occurring variant thereof, for use in therapeutic treatment of a human or non-human aninal is also envisaged as constituting an additional aspect of the invention. Such a polynucleotide will find use in treatment of diseases associated with upregulation of HuIFRG46/ADIR protein.

Polynucleotides of the invention extend to sets of primers for nucleic acid amplification which target sequences within the cDNA for a polypeptide of the invention, e.g. pairs of primers for PCR amplification. In particular, such primers may target regions within the sequences of SEQ ID Nos 1 or 3 or the complementary sequences thereto. The invention also provides probes suitable for targeting a sequence within a cDNA or RNA for a polypeptide of the invention which may be labelled with a revealing label, e.g. a radioactive label or a non-radioactive label such as an enzyme or biotin. In particular, such probes should be suitable for the selective detection of the polynucleotides of the invention, such as the polynucleotide given in SEQ ID Nos 1 or 3 or the complementary sequences thereto. Such probes may be attached to a solid support. Such a solid support may be a micro-array (also commonly referred to as nucleic acid, probe or DNA chip) carrying probes for further nucleic acids, e.g. mRNAs or amplification products thereof corresponding to other Type 1 interferon upregulated genes, e.g. such genes identified as upregulated in response to oromucosal or intravenous administration of IFN-α. Methods for constructing such micro-arrays are well-known (see, for example, EP-B 0476014 and 0619321 of Affymax Technologies N.V. and Nature Genetics Supplement January 1999 entitled “The Chipping Forecast”).

The nucleic acid sequence of such a primer or probe will preferably be at least 10, preferably at least 15 or at least 20, for example at least 25, at least 30 or at least 40 nucleotides in length. It may, however, be up to 40, 50, 60, 70, 100 or 150 nucleotides in length or even longer.

Another aspect of the invention is the use of probes or primers of the invention to identify mutations in HuIFRG46/ADIR genes, for example single nucleotide polymorphisms (SNPs).

As indicated above, in a still further aspect the present invention provides a method of identifying a compound having immunomodulatory activity and/or antiviral activity and/or anti-tumour activity comprising providing a cell capable of expressing HuIFRG46/ADIR protein or a naturally-occurring variant thereof, incubating said cell with a compound under test and monitoring for upregulation of HuIFRG46/ADIR gene expression. Such monitoring may be by probing for mRNA encoding HuIFRG46/ADIR protein or a naturally-occurring variant thereof. Alternatively antibodies or antibody fragments capable of specifically binding one or more of HuIFRG46/ADIR and naturally-occurring variants thereof may be employed.

Antibodies

According to another aspect, the present invention also relates to antibodies (for example polyclonal or preferably monoclonal antibodies, chimeric antibodies, humanised antibodies and fragments thereof which retain antigen-binding capability) which have been obtained by conventional techniques and are specific for a polypeptide of the invention. Such antibodies could, for example, be useful in purification, isolation or screening methods involving immunoprecipitation and may be used as tools to further elucidate the function of HuIFRG46/ADIR protein or a variant thereof. They may be therapeutic agents in their own right. Such antibodies may be raised against specific epitopes of proteins according to the invention. An antibody specifically binds to a protein when it binds with high affinity to the protein for which it is specific but does not bind or binds with only low affinity to other proteins. A variety of protocols for competitive binding or immunoradiometric assays to determine the specific binding capability of an antibody are well-known.

Pharmaceutical compositions

A polypeptide of the invention is typically formulated for administration with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methyl cellulose, carboxymethylcellulose or polyvinyl pyrrolidone; desegregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methyl cellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusions may contain as carrier, for example, sterile water or preferably they may be in the form of sterile, aqueous, isotonic saline solutions.

A suitable dose of HuIFRG46/ADIR protein or a functional analogue thereof for use in accordance with the invention may be determined according to various parameters, especially according to the substance used; the age, weight and condition of the patient to be treated; the route of administration; and the required regimen. Again, a physician will be able to determine the required route of administration and dosage for any particular patient. A typical daily dose may be from about 0.1 to 50 mg per kg, preferably from about 0.1 mg/kg to 10 mg/kg of body weight, according to the activity of the specific inhibitor, the age, weight and condition of the subject to be treated, and the frequency and route of administration. Preferably, daily dosage levels may be from 5 mg to 2 g.

A polynucleotide of the invention suitable for therapeutic use will also typically be formulated for administration with a pharmaceutically acceptable carrier or diluent. Such a polynucleotide may be administered by any known technique whereby-expression of the desired polypeptide can be attained in vivo. For example, the polynucleotide may be introduced by injection, preferably intradermally, subcutaneously or intramuscularly. Alternatively, the nucleic acid may be delivered directly across the skin using a particle-mediated delivery device. A polynucleotide of the invention suitable for therapeutic nucleic acid may alternatively be administered to the oromucosal surface for example by intranasal or oral administration.

A non-viral vector of the invention suitable for therapeutic use may, for example, be packaged into liposomes or into surfactant containing vector delivery particles. Uptake of nucleic acid constructs of the invention may be enhanced by several known transfection techniques, for example those including the use of transfection agents. Examples of these agents include cationic agents, for example calcium phosphate and DEAE dextran and lipofectants, for example lipophectam and transfectam. The dosage of the nucleic acid to be administered can be varied. Typically, the nucleic acid will be administered in the range of from 1 pg to 1 mg, preferably from 1 pg to 10 μg nucleic acid for particle-mediated gene delivery and from 10 μg to 1 mg for other routes.

According to the present invention, HuIFRG46/ADIR polypeptides, polynucleotides or variants of either thereof may be co-administered with other agents or treatments. For the prevention and/or treatment of cancer, a combination of HuIFRG46/ADIR and an anticancer agent or treatment may be administered. For example, the HuIFRG46/ADIR and the anticancer agent or treatment may be administered simultaneously, in the same pharmaceutical formulation or in different pharmaceutical formulations, separately, in formulations which may be administered at the same time or at different times; i.e. sequentially.

Thus, according to the present invention there is provided a product containing a polypeptide or polynucleotide of the invention together with an anti-cancer drug, which product is suitable for use as a combined preparation for simultaneous, separate or sequential use in cancer therapy. The invention also provides methods for the use of such a combined preparation in cancer therapy.

A suitable administration protocol may be determined by a physician and will depend on a number of factors, such as the stability of the formulation(s), the time scale of activity of the drugs or treatments and any potential side-effects. The co-administration of HuIFRG46/ADIR with anticancer drugs or treatments has the advantage that these anticancer treatments may be used in doses which would be sub-effective in sole administration, and are sub-toxic to the patient. Suitable anticancer drugs or treatments may include genotoxic anticancer drugs such as 5-fluorouracil (5-FU), cis-platinum or γ-irradiation.

For anti-viral treatment, HuIFRG46/ADIR polypeptides, polynucleotides or variants thereof, may be administered alone of in combination with other anti-viral treatments. For example, HuIFRG46/ADIR may be used in combination with an interferon such as IFN-α. The two treatments may be administered separately, simultaneously or sequentially.

Prediction of Type 1 interferon responsiveness

As also indicated above, in a still further aspect the present invention provides a method of predicting responsiveness of a patient to treatment with a Type 1 interferon, e.g. IFN-α treatment such as IFN-α treatment by an oromucosal route or intravenously, which comprises determining the level of HuIFRG46/ADIR protein or a naturally-occurring variant thereof, or the corresponding mRNA, in a cell sample from said patient, wherein said sample is taken from said patient following administration of a Type 1 interferon or is treated prior to said determining with a Type 1 interferon in vitro.

Preferably, the Type 1 interferon for testing responsiveness will be the Type 1 interferon selected for treatment. It may be administered by the proposed treatment route and at the proposed treatment dose. Preferably, the subsequent sample analysed may be, for example, a blood sample or a sample of peripheral blood mononuclear cells (PBMCs) isolated from a blood sample.

More conveniently and preferably, a sample obtained from the patient comprising PBMCs isolated from blood may be treated in vitro with a Type 1 interferon, e.g. at a dosage range of about 1 to 10,000 IU/ml. Such treatment may be for a period of hours, e.g. about 7 to 8 hours. Preferred treatment conditions for such in vitro testing may be determined by testing PBMCs taken from normal donors with the same interferon and looking for upregulation of an appropriate expression product. Again, the Type 1 interferon employed will preferably be the Type 1 interferon proposed for treatment of the patient, e.g. recombinant IFN-α. PBMCs for such testing may be isolated in conventional manner from a blood sample using Ficoll-Hypaque density gradients. An example of a suitable protocol for such in vitro testing of Type 1 interferon responsiveness is provided in Example 3 below.

The sample, if appropriate after in vitro treatment with a Type 1 interferon, may be analysed for the level of HuIFRG46/ADIR protein or a naturally-occurring variant thereof. This may be done using an antibody or antibodies capable of specifically binding one or more of HuIFRG46/ADIR protein and naturally-occurring variants thereof, e.g. allelic variants thereof. Preferably, however, the sample will be analysed for mRNA encoding HuIFRG46/ADIR protein or a naturally-occurring variant thereof. Such mRNA analysis may employ any of the techniques known for detection of mRNAs, e.g. Northern blot detection or mRNA differential display. A variety of known nucleic acid amplification protocols may be employed to amplify any mRNA of interest present in the sample, or a portion thereof, prior to detection. The mRNA of interest, or a corresponding amplified nucleic acid, may be probed for using a nucleic acid probe attached to a solid support. Such a solid support may be a micro-array as previously discussed above carrying probes to determine the level of further mRNAs or amplification products thereof corresponding to Type 1 interferon upregulated genes, e.g. such genes identified as upregulated in response to oromucosal or intravenous administration of IFN-α.

The following examples illustrate the invention:

### EXAMPLES

### Example 1

Previous experiments had shown that the application of 5 μl of crystal violet to each nostril of a normal adult mouse using a P20 Eppendorf micropipette resulted in an almost immediate distribution of the dye over the whole surface of the oropharyngeal cavity. Staining of the oropharyngeal cavity was still apparent some 30 minutes after application of the dye. These results were confirmed by using ¹²⁵I-labelled recombinant human IFN-α1-8 applied in the same manner. The same method of administration was employed to effect oromucosal administration in the studies which are described below.

Six week old, male DBA/2 mice were treated with either 100,000 IU of recombinant murine interferon α (IFN α) purchased from Life Technologies Inc, in phosphate buffered saline (PBS), 10 μg of recombinant human interleukin 15 (IL-15) purchased from Protein Institute Inc, PBS containing 100 μg/ml of bovine serum albumin (BSA), or left untreated. Eight hours later, the mice were sacrificed by cervical dislocation and the lymphoid tissue was removed surgically from the oropharyngeal cavity and snap frozen in liquid nitrogen and stored at −80° C. RNA was extracted from the lymphoid tissue by the method of Chomczynski and Sacchi 1987, (Anal. Biochem. 162, 156-159) and subjected to mRNA Differential Display Analysis (Lang, P. and Pardee, A. B., Science, 257, 967-971).

Differential Display Analysis

Differential display analysis was carried out using the “Message Clean” and “RNA image” kits of the GenHunter Corporation essentially as described by the manufacturer. Briefly, RNA was treated with RNase-free DNase, and 1 μg was reverse-transcribed in 100 μl of reaction buffer using either one or the other of the three one-base anchored oligo-(dT) primers A, C, or G. RNA was also reverse-transcribed using one or the other of the 9 two-base anchored oligo-(dT) primers AA, CC, GG, AC, CA, GA, AG, CG, GC. All the samples to be compared were reverse transcribed in the same experiment, separated into aliquots and frozen. The amplification was performed with only 1 μl of the reverse transcription sample in 10 μl of amplification mixture containing Taq DNA polymerase and α-³³P dATP (3,000 Ci/mmole). Eighty 5′ end (HAP) random sequence primers were used in combination with each of the (HT11) A, C, G, AA, CC, GO, AC, CA, GA, AG, CG or GC primers. Samples were then run on 7% denaturing polyacrylamide gels and exposed to autoradiography. Putative differentially expressed bands were cut out and reamplified according to the instructions of the supplier. A 125 bp differentially expressed cDNA was identified and characterised as the terminus of the 3′UTR of a previously unrecognised transcript with a typical polyadenylation site located 14 bp upstream of the polyA tail. The novel cDNA was used to probe a Northern blot of RNA from the oropharyngeal tissue of IFN treated, IL-15 treated, and excipient treated animals.

Two RNA transcripts of 1.9 and 3.5 kb were identified in samples form IFN treated animals. Two similar transcripts were also detected at high levels in the liver and spleen of mice following intraperitoneal administration of the same dose of IFNα. The same two bands were also observed in tissue from control mice upon over-exposure of the blots. An additional weak 2.4 kb band was also detected in the spleen and other organs from IFN treated animals upon overexposure of the blots.

Cloning and Sequencing

Briefly, re-amplified bands from the differential display screen were cloned in the Sfr 1 site of the pPCR-Script SK(+) plasmid (Stratagene) and cDNAs amplified from the rapid amplification of cDNA ends were isolated by TA cloning in the pCR3 plasmid (Invitrogen). DNA was sequenced using an automatic di-deoxy sequencer (Perkin Elmer ABI PRISM 377).

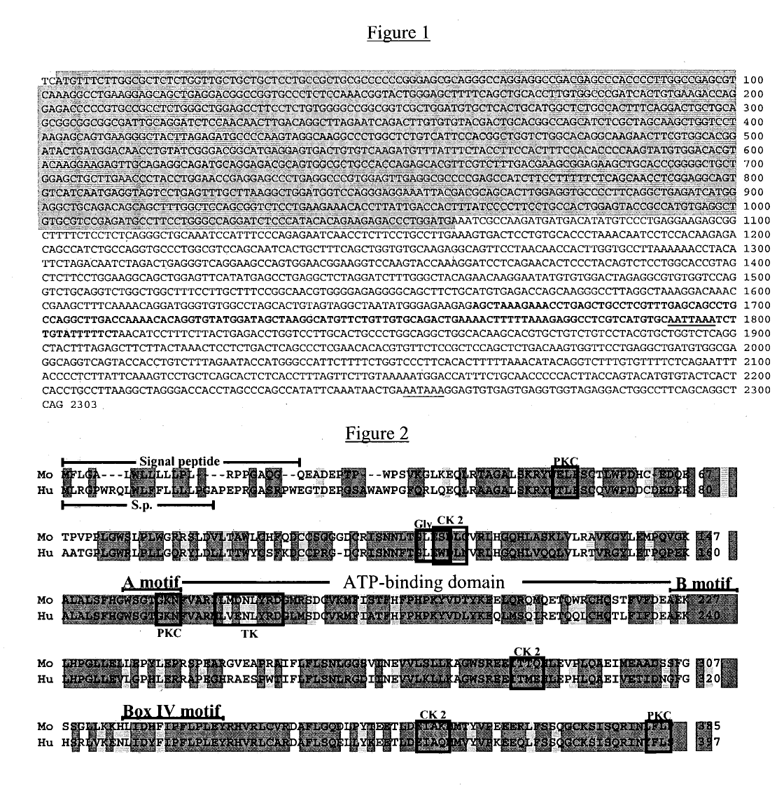

The 125 bp cDNA was used as a probe to screen a cDNA library from the spleen of BALB/c mice. Thirteen overlapping cDNAs were obtained from 10⁶ lambda phage and their sequences were combined to form a contig of 2300 bp (FIG. 1). This sequence contained an open reading frame (ORF) of 1158 bp encoding a novel protein with a deduced molecular weight of 44 kDa (FIG. 2). The deduced amino acid sequence contained a potential signal peptide of 21 amino acids and a characteristic Walker ATP/Mg²⁺binding site (Walker et al (1982) EMBO J 1: 945-951) identified by the presence of typical A, B, and Box IV motifs, and by homology with the ATP binding domain of other well characterised proteins (Ozelius et al (1997) Nat Genet 17: 40-48; Ozelius et al (1998) Adv Neurol 78: 93-105; Schirmer et al (1996) Trends Biochem Sci 8: 289-296).

The 2300 bp contig encompassed the sequence of the differentially expressed 125 bp cDNA band at positions 1662-1810 (FIG. 1, bold). The 3′ terminus of the differentially expressed band is believed to correspond to the 3′ end of the 1.9 kb transcript identified by Northern blot. The use of a second potential polyadenylation site, detected at position 2253-2258 may give rise to the less represented 2.4 kb transcript. The contig constructed from the phage sequences was extended toward the 3′ end by the addition of overlapping EST sequences from the Genbank/EMBL dbest database. This longer sequence contained a typical AATAAA polyadenylation site at position 3250 bp. These observations suggest that the 3.5 kb message identified on Northern blots represents the continuation of the shorter mRNA to this second polyadenylation site.

In order to determine whether the increase in HuIFRG46/ADIR mRNA following IFN treatment reflected the presence of an interferon sensitive response element (ISRE) in the HuIFRG46/ADIR promoter, 800 bp of the 5′ flanking region of the HuIFRG46/ADIR gene was cloned using a genome walking approach, sequenced (AJ318043), and shown to contain the sequence GAGTTTCATTTCGGA at positions −185 to −171 upstream from the start site of the mRNA sequence, which fits well with a potential binding site for ISGF3, the principal transcriptional activator of IFN stimulated genes.

Isolation of Human cDNA

Differentially expressed murine 3′ sequences identified from the differential display screen were compared with random human expressed sequence tags (EST) present in the dbEST database of GenBank™ of the United States National Center for Biotechnology Information (NCBI). The sequences potentially related to the murine EST isolated from the differential display screen were combined in a contig and used to construct a human consensus sequence corresponding to a putative cDNA. A full length cDNA was then generated by RT-PCR from RNA extracted from Daudi cells, cloned and sequenced. The human cDNA was found to be 1285 nucleotides in length. This corresponded to the mouse gene whose expression was found to be enhanced approximately 5-fold in the lymphoid tissue of the oral cavity of mice following oromucosal administration of IFN-α.

A unique cDNA fragment of the predicted size was obtained, cloned and sequenced (SEQ. ID. No.1). This human cDNA contains an open reading frame (ORF) of 1194 bp in length at positions 74-1267 encoding a protein of 397 amino acids (SEQ. ID. No. 2) with a calculated molecular weight of 46 kDa and has been localised on human chromosome 1. The human and mouse cDNA sequences exhibit 85% identity and the proteins which they encode are 70% identical and display similar features including includes a hydrophobic N-terminal sequence and an ATP binding domain with typical A, B and Box IV motifs. Also conserved are eight potential phosphorylation sites and an N-glycosylation site.

A number of different clones obtained by the above method were sequenced. The majority had the nucleotide sequence shown in SEQ. ID. No. 1. Some clones had the nucleotide sequence show in SEQ. ID. No. 3. The nucleotide sequence shown in SEQ. ID. No. 3 differs at positions 110 and 1256 from the sequence shown in SEQ. ID. No. 1. The protein encoded by SEQ. ID. No. 3 is shown in SEQ. ID. No. 4. This protein differs from that shown in SEQ. ID. No. 2 at positions 13 and 395.

Comparison of the promoter region of the mouse gene with the corresponding human HuIFRG46/ADIR sequence on chromosome 1 revealed a clear homology with a number of highly conserved boxes, although no sequence homologous to the putative mouse ISRE was identified in the human gene. These observations may explain the decreased sensitivity of the human gene to interferon treatment compared with the mouse gene.

### Example 2

Administration of IFN-α

Male DBA/2 mice were injected intraperitoneally (ip) or oromucosally (om) with 100,000 IU of recombinant murine IFN-α purchased from Life Technologies Inc. For ip administration, animals were treated with 100,000IU IFN-α in 200 μl of PBS or treated with an equal volume of PBS alone. For om administration, 10 μl of IFN-α or excipient was applied with a 1 to 20 μl capacity adjustable micropipette to the nostrils of the mice. Four hours after ip or om treatment, the animals were sacrificed by cervical dislocation and tissue from the oral cavity and the spleen were removed using conventional procedures. Total RNA was extracted by the method of Chomczynski and Sacchi (Anal. Biochem. (1987) 162,156-159) and 10.0 μg of total RNA per sample was subjected to Northern blotting in the presence of glyoxal and hybridised with a cDNA probe for HuIFRG46/ADIR mRNA as described by Dandoy-Dron et al.(J. Biol. Chem. (1998) 273, 7691-7697). The blots were first exposed to autoradiography and then quantified using a Phospholmager according to the manufacturer's instructions.

Two RNA transcripts of 1.9 and 3.5 kb were identified in the samples from IFN treated animals. The same two bands were also observed in tissue from control mice upon over-exposure of the blots. Quantitative analysis showed that HuIFRG46/ADIR RNA transcripts were increased approximately 5 fold to 6 fold in tissue from the oropharyngeal cavity four hours after treatment, and approximately 8 and 15 fold in the spleen and liver respectively four hours after ip treatment.

### Example 3

Administration of ECMV

Six week old Swiss mice were infected with the neurotropic virus Encephalomyocarditis virus (ECMV). ECMV strain JH was propagated on murine L929 cells using standard methods as described in Gresser et al (1968) (Proc. Soc, Exp. Biol. Med 127: 491-496). The virus stock used had a titer of 2.1×10⁹ TCID 50 on murine L919 cells. Mice were infected with approximately 100 LD50 of EMCV in a volume of 200 μl Dulbecco's minimal essential medium +2% fetal bovine serum by intraperitoneal injection. Four hours after infection, mice were sacrificed by cervical dislocation and RNA extracted as described in Example 2. Infection with EMCV resulted in a 20 to 40 fold increase in the level of the new HuIFRG46/ADIR mRNA transcripts in the brains of terminally ill animals compared to uninfected controls. In contrast, the level of scrapie responsive gene one (Scrg1) mRNA was not modified by ECMV infection.

### Example 5

Expression of HuIFRG46/ADIR mRNA

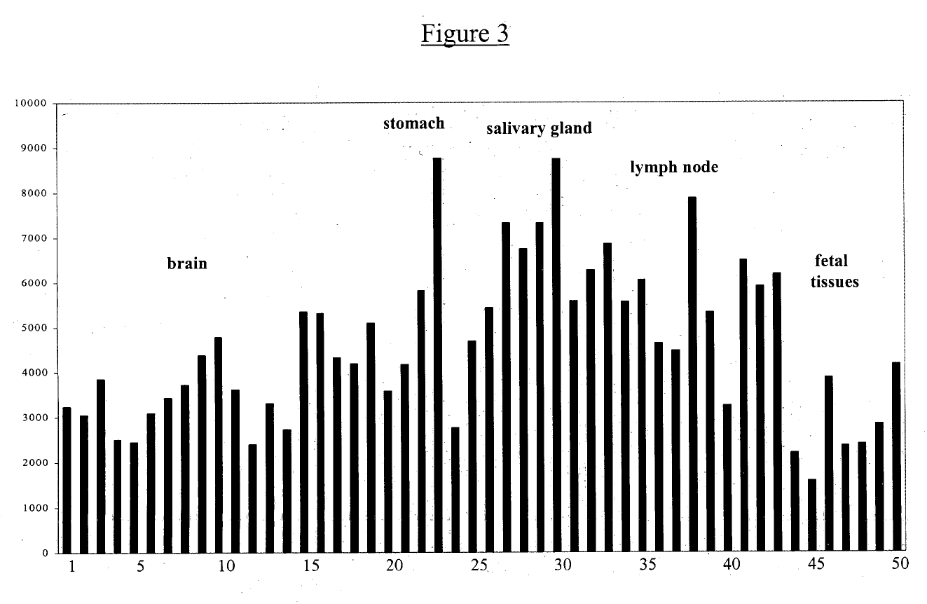

The HuIFRG46/ADIR coding sequence was amplified and used as a probe to determine the tissue distribution of HuIFRG46/ADIR mRNA and the effect of IFN treatment on its expression. A membrane to which poly A⁺ RNA from fifty human tissues had been immobilized in separate dots was hybridized with the human HuIFRG46/ADIR cDNA according to the instructions of the supplier, and the membrane was subjected to phosphoimaging quantification. A master blot containing RNA from 50 different human tissues was analysed. HuIFRG46/ADIR transcripts were detected in all the adult and fetal organs analysed and were most abundant in the stomach, salivary glands and lymph nodes (see FIG. 3).

A multi-tissue Northern blot was hybridised with a radiolabelled probe derived form the HuIFRG46/ADIR coding sequence. One HuIFRG46/ADIR mRNA species of 2,3 kb was detected in all the human tissues. An additional 1.25 kb mRNA transcript was detected in placenta A Northern blot containing the same amount of RNA from a range of human tissues was hybridized with a probe specific for the coding sequence of HuIFRG46/ADIR 1 or with a probe specific for the alternatively used exon 7. The exon 7 probe identifies a 1.25 kb transcript encoding a smaller protein termed ADIR2. The blot was hybridized with a probe specific for the coding sequence of HuIFRG46/ADIR. A G3PDH probe was used for normalization of the Phospholmager quantification of the bands.

Alignment of the RT-PCR amplified HuIFRG46/ADIR cDNA with the genomic sequence of chromosome 1 showed that normal HuIFRG46/ADIR mRNA results from the transcription of 6 exons and that the principal protein motifs are encoded by different exons. A putative alternative splicing site was identified, using the downstream exon 7 (196 bp) rather than the usual exon 6 (1025 bp). This would generate a shorter 1.15 kb transcript terminating in a typical polyadenylation site, the size of which corresponds to the shorter message identified in placenta. A 200 bp cDNA was amplified by RT_PCR from placental RNA using primers specific for exon 5 and exon 7. Cloning and sequencing of this fragment revealed a direct exon 5-exon 7 junction confirming that alternative splicing of the HuIFRG46/ADIR gene can occur. Hybridisation of a probe specific for exon 7 to the multi-tissue Northern blot revealed the 1.25 kb mRNA only in placenta. This transcript encodes a shorter protein of 337 amino acids, denominated HuIFRG46/ADIR2 which lacks the box IV motif present in HuIFRG46/ADIR (FIG. 4b).

### Example 4

Testing Type 1 interferon responsiveness in vitro

HeLa cells were treated in vitro with 10,000 IU of recombinant human IFN-α2 (Intron A from Schering-Plough) in PBS or with an equal volume of PBS alone. Eight hours later the cells were centrifuged (800×g for 10 minutes) and the cell pellet recovered. Total RNA was extracted from the cell pellet by the method of Chomczynski and Sacchi and 10.0 μg of total RNA per sample was subjected to Northern blotting in the presence of glyoxal and hybridised with a cDNA probe for HuIFRG46/ADIR mRNA as previously described in Example 2 above. Enhanced levels of mRNA for HUIFRG46/ADIR protein (approximately 10-fold) were detected in samples of RNA extracted from IFN-α treated HeLa cells compared to samples treated with PBS alone. Using the same methods, HUIFRG46/ADIR RNA also increased 11 and 4 fold in mouse L929 cells treated in vitro with 10⁴ IU of IFN-α or IFN-γ respectively, and 2 to 3 fold in human Daudi cells treated with 10⁴ IU of IFN-α in vitro. In a further experiment, treatment of HeLa, MRC5 and HuH7 cells with IFNα or IFNγ did not modify significantly HuIFRG46/ADIR mRNA levels.

The same procedure may be used to predict Type 1 interferon responsiveness using human peripheral blood mononuclear cells PBMCs taken from a patient proposed to be treated with a Type 1 interferon. PBMCs are isolated on Ficoll-Hypaque density gradients and treated in vitro with 10,000 IU of recombinant human IFN-α2 (intron A from Schering-Plough) in PBS or with an equal volume of PBS alone. Eight hours later the cells are centrifuged (800×g for 10 minutes) and the cell pellet recovered. Total RNA is extracted from the cell pellet and 10 μg of RNA per sample is subjected to Northern blotting as described above.

### Example 5

Homology of HuIFRG46/ADIR with known genes and proteins

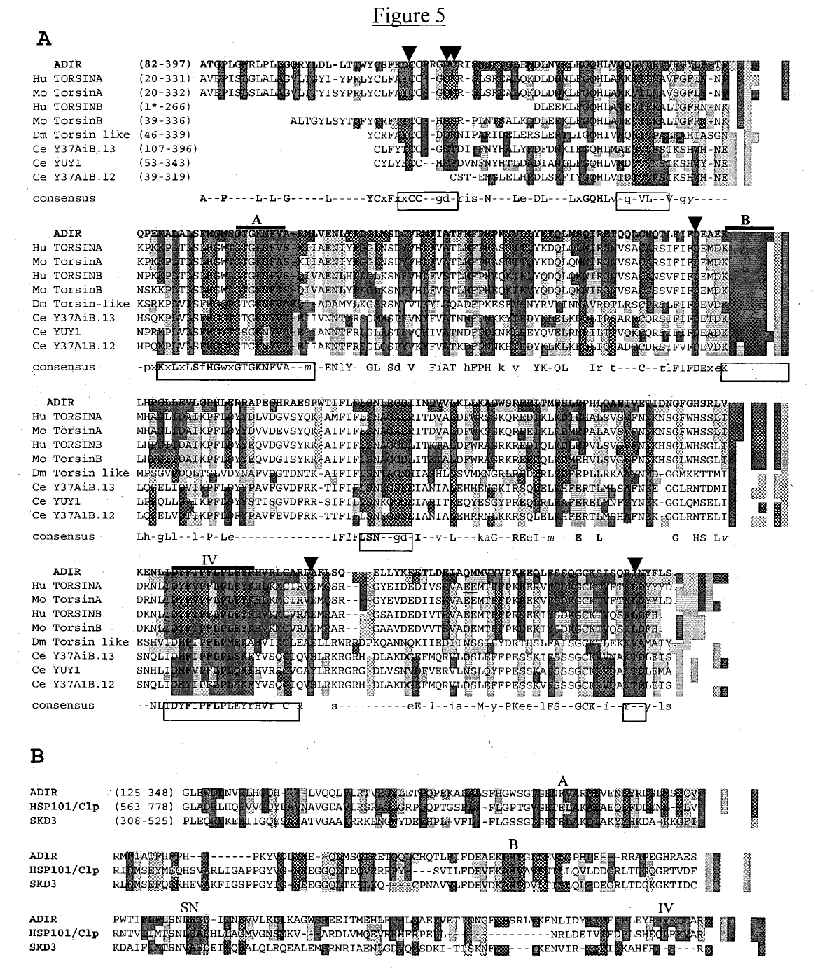

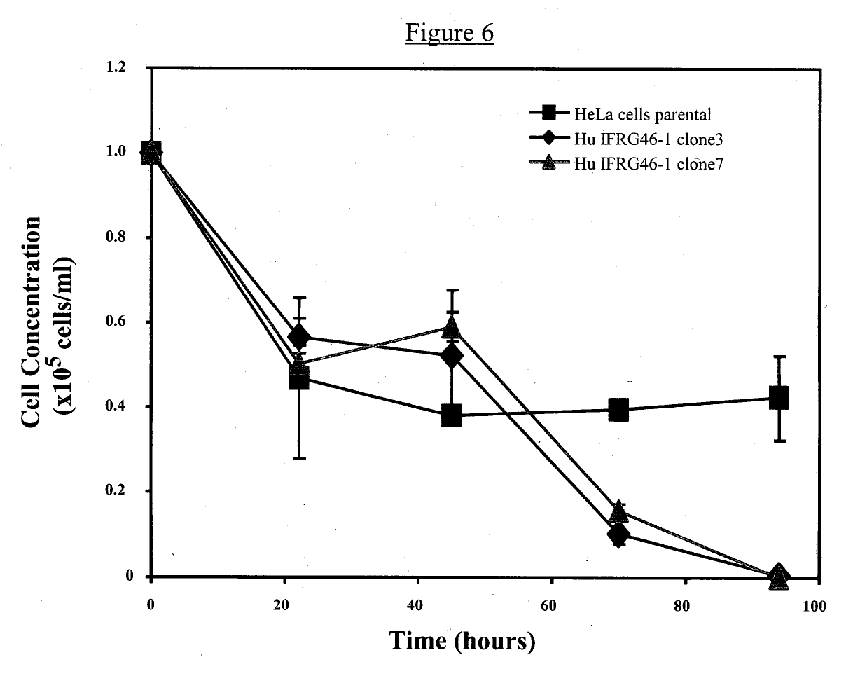

A search of the Genbank/EMBL protein databases revealed a high degree of homology between HuIFRG46/ADIR and five sequences corresponding to Torsin A and B from different mammalian species, including man, monkey, and the mouse. HuIFRG46/ADIR also exhibited a slightly lower degree of homology with four Torsin-like sequences; three from the nematode C. elegans and one from the fruit-fly Drosophila melanogaster. The homology of HuIFRG46/ADIR with the Torsin family is remarkably high and extensive, with 38% of identical amino acids and 60% similarities to Torsin A in a 309 bp alignment (FIG. 5). The homologous region covered almost the whole of the Torsin A sequence. Similar homology was observed with the closely related Torsin B protein. Torsin A and B cDNAs were recently isolated by mapping and positional cloning (Ozelius et al (1997) Nat Genet 17: 40-48; Dron et al (1999) Arch Virol 144: 19-28; Ozelius et al (1999) Genomics 62: 377-384) and shown to encode ATP binding proteins the function of which remain unknown. It is important to note that despite the high degree of homology of HuIFRG46/ADIR with Torsin A and B proteins, their nucleotide sequences share no consistent homology. Thus, HUIFRG46/ADIR is either unrelated or at best distantly related to the Torsins genes but encodes a product with sequence homology and possibly structural and functional similarities to Torsins.

Torsin proteins have been reported previously to contain a putative ATP binding site comprising four characteristic motifs (Ozelius et al (1997) Nat Genet 17: 40-48; Dron et al (1999) Arch Virol 144:19-28); the canonical Walker A and B nucleotide and Mg²⁺-binding elements, a conserved SN motif, and a carboxy-terminal signature sequence, the Box IV motif. The sequences of all four motifs were found to be highly conserved in the HuIFRG46/ADIR protein. We also identified other blocks of sequence homology, more specific to the homology between HuIFRG46/ADIR and Torsin A and B per se: YCxFxxCC (position in HuIFRG46/ADIR 105-112), LxGQHL (position 114-139), and GCK (position 384-386). These three later amino acid boxes are also present in the Torsin-like sequences of C. elegans and Drosophila melanogaster. Another remarkable feature is the position of six cysteine residues distributed throughout the HuIFRG46/ADIR sequence which are also conserved in Torsin A, Torsin B, and all the other members of the Torsin family. Moreover a potential phosphokinase C phosphorylation site associated with the A motif consensus sequence (LSxHGWsGTGKNFV), is fully conserved in HuIFRG46/ADIR and the Torsin proteins. A potential Tyrosine Kinase phosphorylation site is also conserved just after the A motif in HuIFRG46/ADIR and Torsin A, but not in Torsin B.

HuIFRG46/ADIR was also found to exhibit weak but consistent homology with members of the Clp/ISP100 family of chaperone-like (Schirmer et al (1996) Trends Biochem Sci 8: 289-296) which belong to the large family of ATPases associated with a variety of cellular activities (AAA) (Neuwald et al (1999) Genome Res 9: 27-43; Confalonieri & Duguet (1995) Bioessays 17: 639-650). HUIFRG46/ADIR exhibits 20 to 30% identity (40 to 50% similarity) in an alignment of 200 to 270 amino acids with three different classes of bacterial ATP binding chains, CipA, ClpB and ClpC and with the yeast heat shock proteins HSP78, HSP101, and HSP104. Most of the Clp/HSP100 members are large proteins and the homology that they share with HuIFRG46/ADIR is principally in the region involved in ATP binding. HuIFRG46/ADIR also exhibited a weak degree of homology with SKD3 (Perier et al (1995) Gene 152: 157-163) a mammalian protein containing an ATP-binding domain similar to that of the Clp/Hsp 100 family. HuIFRG46/ADIR contains one predicted ATP-binding domain only and thus may belong to the Class 2 sub-family of the Clp/HSP100 proteins.

Given the central role played by interferons in regulating the expression of genes critical for antigen presentation and immune surveillance against viruses and tumor cells, these data suggest that HuIFRG46/ADIR may be an ATPase involved in protein processing in the endoplasmic reticulum. Such proteins are often involved in the processes which lead to apoptosis, a process which is known to play an important role in the antitumor activity of the interferons and indirectly in their antiviral activity since viruses require viable cells in which to replicate.

### Example 6

Expression of HUIFRG46/ADIR-HAT and HUIFRG46/ADIR-EGFP fusion proteins in HeLa cells.

The histidine affinity Tag (HAT) of the plasmid PHAT10/11/12 (Clontech, Palo Alto, Calif.) containing six histidine residues separated by three other amino acids, was PCR amplified, fused in phase with the entire coding sequence of the HuIFRG46/ADIR cDNA and inserted into the expression vector pLNCX2 (Clontech, Palo Alto, Calif.) for retrovirus mediated gene transfer using the pan-tropic retroviral system developed by Clontech Laboratories according to the manufacturer's instructions. Stable transfected clones of HeLa cells were selected in the presence of G418 (Gibco-BRL, Rockville, AM, USA). Crude cellular extracts were then separated by 12% SDS-polyacrylamide gel electrophoresis, transferred to PVDF filters, and immunoblotted with a rabbit anti-HAT polyclonal antibody at a 1/10,000 dilution, and further incubated with a peroxidase-conjugated goat anti-rabbit IgG (Jackson Immunoresearch Laboratories, MA) at 1/20,000 dilution. The protein was detected by enhanced chemoilluminescence according to the manufacturer's instructions (Amersham Pharmacia, UK). Cellular extracts of two clones (clones 3 and 7) were analysed by Western blot and were shown to produce high levels of a protein of 50 kDa, the predicted size of HuIFRG46/ADIR protein. These clones were then tested in parallel with untransfected parental HeLa cells or HeLa cells transfected with HAT-PHAT10/11/12 vector alone.

HeLa cells were also transfected with an HUIFRG46/ADIR-EGFP fusion protein. The HUIFRG46/ADIR-EGFP fusion protein was expressed throughout the cytoplasm and co-localized with protein disulfide-isomerase (PDI) a marker of the ER. Similarly, the fusion protein also exhibited a high degree of co-localization with a co-transfected ECFP-ER fusion protein which contained the ER targeting sequence of calreticulin and localized in the lumen of the ER. Similar results were obtained for both transiently transfected HeLa cells and for clones of stable transfectants.

### Example 7

Effect of HuIFRG46/ADIR on human tumor cells

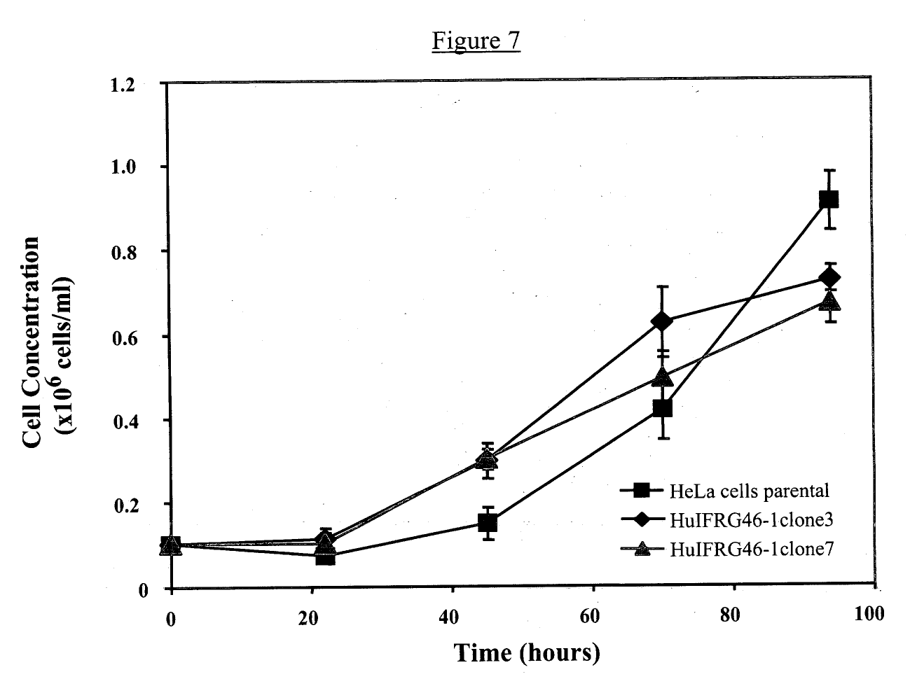

Parental HeLa cells or HeLa cells transfected with the HuIFRG46/ADIR HAT-PHAT10/11/12 vector expressing the HuIFRG46/ADIR protein (clones 3 and 7) were seeded in 96 well microtiter plates at a concentration of 10⁵ cells in DMEM medium containing 10% fetal bovine serum in the presence or absence of 5 μm 5-fluorouracil (5-FU, Sigma-Aldrich, St Louis, Mo.). Cell proliferation was then followed daily using a haemocytometer and the trypan blue dye exclusion test to distinguish between viable and dead cells according to standard procedures. FIG. 6 shows that HuIFRG46/ADIR induces massive apoptosis of human tumor cells in the presence of 5-FU. Indeed, all the tumor cells were killed in the presence of HuIFRG46/ADIR and 5-FU, even though 5-FU in the concentration used (5 μM) alone has no significant effect on the proliferation or apoptosis of human tumor cells. Although HuIFRG46/ADIR alone had no significant effect on the apoptosis of human tumor cells, a statistically significant inhibition of cell proliferation was observed after 96 hours cultivation of cells in the presence of HuIFRG46/ADIR (FIG. 7).

### Example 8

Antiviral activity of HuIFRG46/ADIR

Parental HeLa cells or HeLa cells transfected with the HuIFRG46/ADIR HAT-PHAT10/11/12 vector expressing the HuIFRG46/ADIR protein (clone-7) were seeded in 96 well microtiter plates at a concentration of 10⁵ cells in DMEM medium containing 2% fetal bovine serum and incubated overnight at 37° C. in the presence or absence of 100 IU of IFN-α. The IFN containing medium was then removed and the cells were infected with vesicular stomatitis virus (VSV) at a multiplicity of infection of 0.01 in DMEM medium containing 2% fetal bovine serum. One hour later the virus was removed and the cultures were washed three times with DMEM medium containing 2% fetal bovine serum and then incubated overnight at 37° C. The cultures were then frozen and thawed six times, centrifuged for 10 minutes at 15,000×g to remove cell debris and the virus yield was determined by titration of ten fold dilutions of each of the culture supernatant on L929 cells using standard procedures.

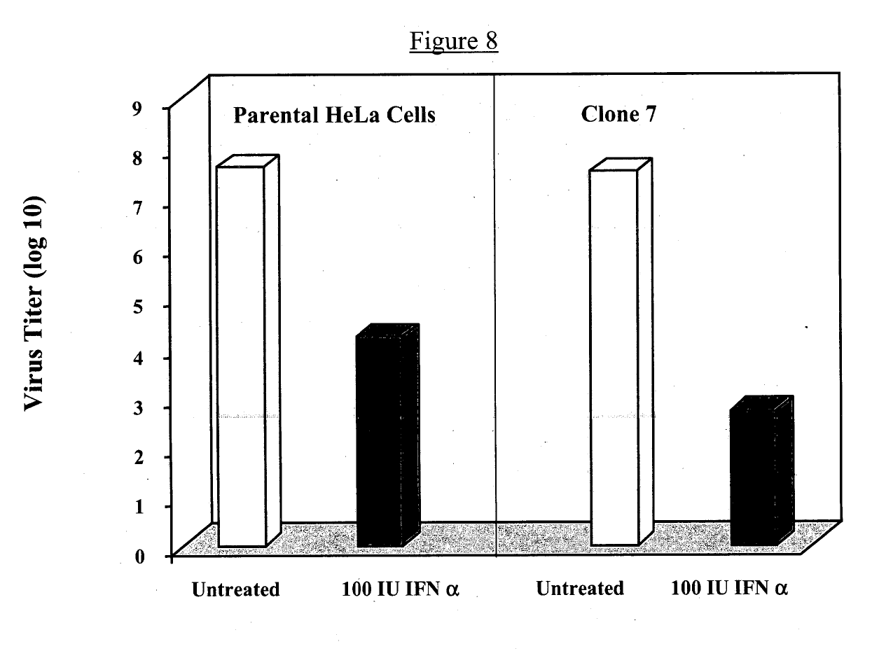

HuIFRG46/ADIR was found to markedly potentiate the antiviral activity of IFN-α in cells infected with VSV. The virus yield in HeLa cells infected with VSV at a multiplicity of infection of 0.01 was 6×10⁷ in the untreated control culture and 2×10⁴ in the culture treated with 100 IU of IFN-α (FIG. 8). in contrast, the virus yield in HeLa cells expressing HuIFRG46/ADIR (clone 7) and infected with VSV under the same conditions as parental HeLa cells was 5×10⁷ in the control culture and 7×10² in the culture treated with 100 IU of IFN-α. Thus, HuIFRG46/ADIR was found to potentiate the antiviral activity of 100 IU of IFN-α by approximately 30 fold.

### Example 9

Effect of HuIFRG46/ADIR on human tumor cells in nude mice

Six week old male BALB/c Nu+/Nu+ nude mice were injected subcutaneously with 4×10⁶ parental HeLa cells or HeLa cells (clone 7) expressing HuIFRG46/ADIR. Seven days later when the subcutaneous tumors had reached a size of 26.8+/−1.58 mm² and 22.4+/−2.46 mm² for parental HeLa cells and clone 7 respectively, the animals were treated with 30 mg/kg of 5-FU (a sub-effective dose) in PBS, or with PBS alone, on days 7, 8, 9, 10, 14, 15, 16, 17, 18 and 21. The size of the tumors was determined daily. The animals were then sacrificed on day 29 and the tumors were harvested and processed for histology using standard procedures.

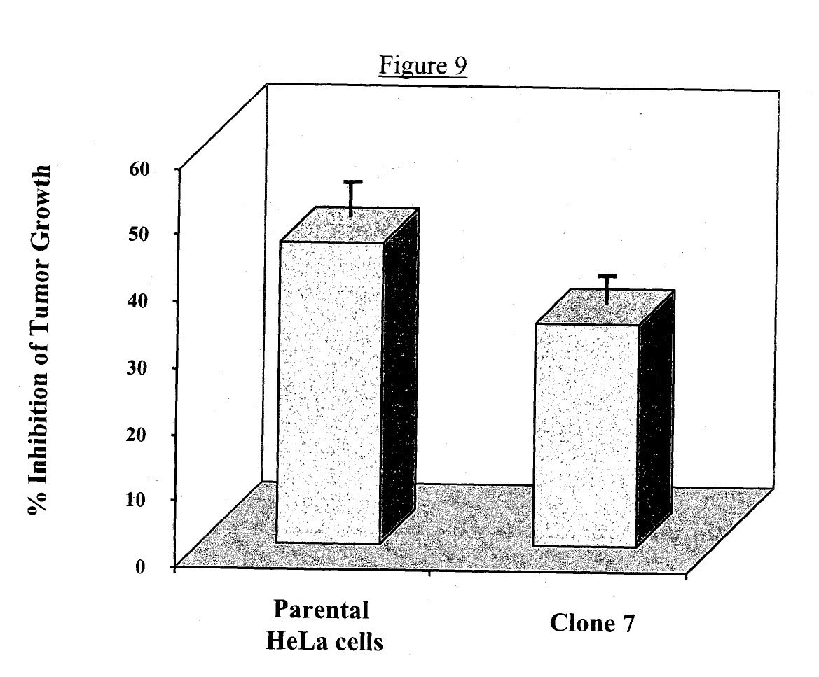

The results presented in FIG. 9 show that the antitumor activity of 5-FU was clearly greater in the presence of HuIFRG46/ADIR.

1. An isolated polypeptide comprising
(i) the amino acid sequence of SEQ ID NO: 2 or of SEQ ID NO: 4;
(ii) a variant thereof having substantially similar function selected from immunomodulatory activity and/or anti-viral activity and/or anti-tumour activity; or
(iii) a fragment of (i) or (ii) which retains substantially similar function selected from immunomodulatory activity and/or anti-viral activity and/or anti-tumour activity.

2. A variant or fragment of the polypeptide defined by the amino acid sequence set forth in SEQ. ID. No. 2 or in SEQ. ID. No. 4 suitable for raising specific antibodies for said polypeptide and/or a naturally-occurring variant thereof.

3. A polynucleotide encoding a polypeptide as claimed in claim 1 or 2.

4. A polynucleotide as claimed in claim 3 which is a cDNA.

5. A polynucleotide encoding a polypeptide as claimed in claim 1, which polynucleotide comprises:
(a) the nucleic acid sequence of SEQ ID NO: 1 or of SEQ ID NO: 3 or the coding sequence thereof and/or a sequence complementary thereto;
(b) a sequence which hybridises to a sequence as defined in (a);
(c) a sequence that is degenerate as a result of the genetic code to a sequence as defined in (a) or (b); or
(d) a sequence having at least 60% identity to a sequence as defined in (a), (b) or (c).

6. An expression vector comprising a polynucleotide sequence as claimed in any one of claims 3 to 5, which is capable of expressing a polypeptide according to claim 1 or 2.

7. A host cell containing an expression vector according to claim 6.

8. An antibody or a fragment thereof which retains antigen-binding capability specific for a polypeptide as claimed in claim 1 or claim 2.

9. An isolated polynucleotide which directs expression in vivo of a polypeptide as claimed in claim 1.

10. A polypeptide as claimed in claim 1 or a polynucleotide as claimed in claim 9 for use in therapeutic treatment of a human or non-human animal.

11. A polypeptide as claimed in claim 1 or a polynucleotide as claimed in claim 9 for use in cancer therapy.

12. A pharmaceutical composition comprising a polypeptide as claimed in claim 1 or a polynucleotide as claimed in claim 9 and a pharmaceutically acceptable carrier or diluent.

13. A product containing both a polypeptide as claimed in claim 1 or a polynucleotide as claimed in claim 9 and an anti-cancer drug, wherein said product is suitable for use as a combined preparation for simultaneous, separate or sequential use in cancer therapy.

14. Use of a polypeptide as claimed in claim 1 or a polynucleotide as claimed in claim 9 in the preparation of medicament for use in therapy as an anti-viral, anti-tumour or immunomodulatory agent.

15. A method of treating a patient having a Type 1 interferon treatable disease, which comprises administering to said patient an effective amount of a polypeptide as claimed in claim I or a polynucleotide as claimed in claim 9.

16. A method of treating a patient having a viral disease, which comprises administering to said patient an effective amount of a polypeptide as claimed in claim 1 or a polynucleotide as claimed in claim 9.

17. A method of treating or preventing cancer in a patient, which comprises administering to said patient an effective amount of a polypeptide as claimed in claim 1 or a polynucleotide as claimed in claim 9.

18. A method of treating or preventing cancer in a patient, which comprises administering to said patient a combined preparation comprising an anti-cancer drug and an effective amount of a polypeptide as claimed in claim 1 or a polynucleotide as claimed in claim 9.

19. A method of producing a polypeptide according to claim 1 or 2, which method comprises culturing host cells as claimed in claim 7 under conditions suitable for obtaining expression of the polypeptide and isolating the said polypeptide.

20. A method of identifying a compound having immunomodulatory activity and/or anti-viral activity and/or anti-tumour activity comprising providing a cell capable of expressing the polypeptide of SEQ. ID. No. 2 or of SEQ. ID. No. 4 or a naturally-occurring variant thereof, incubating said cell with a compound under test and monitoring for upregulation of the gene encoding said polypeptide or variant.

21. A polynucleotide capable of expressing in vivo an antisense sequence to a coding sequence for the amino acid sequence defined by SEQ. ID. No.2 or by SEQ. ID. No. 4 or a naturally-occurring variant of said coding sequence for use in therapeutic treatment of a human or non-human animal.

22. An antibody or fragment as claimed in claim 8 for use in therapeutic treatment.

23. A set of primers for nucleic acid amplification which target sequences within a cDNA as claimed in claim 4, said target sequences being part of a sequence as claimed in claim 5 part (a).

24. A nucleic acid probe derived from a polynucleotide as claimed in any one of claims 3 to 5 which probe is suitable for selective detection of a sequence as claimed in claim 5 part (a).

25. A probe as claimed in claim 24 which is attached to a solid support.

26. A method of predicting responsiveness of a patient to treatment with a Type 1 interferon, which comprises determining the level of the protein defined by the amino acid sequence set forth in SEQ. ID. No. 2 or in SEQ. ID. No. 4 or a naturally-occurring variant thereof, or the corresponding mRNA, in a cell sample from said patient, wherein said sample is obtained from said patient following administration of a Type 1 interferon or is treated prior to said determining with a Type 1 interferon in vitro.

27. A method as claimed in claim 26 wherein the interferon administered prior to obtaining said sample or used to treat said sample in vitro is the interferon proposed for treatment of said patient.

28. A method as claimed in claim 26 or claim 27 wherein a sample comprising peripheral blood mononuclear cells isolated from a blood sample of the patient is treated with a Type 1 interferon in vitro.

29. A method as claimed in any one of claims 26 to 28 wherein said determining comprises determining the level of mRNA encoding the protein defined by the sequence set forth in SEQ. ID. No. 2 or in SEQ. ID. No. 4 or a naturally-occurring variant of said protein.

30. A non-human transgenic animal capable of expressing a polypeptide that is claimed in claim 1.


SEQUENCE LISTING (24 sequences)

SEQ ID NO: 1 - DNA, 1285 bp, Homo sapiens
  CDS (74)..(1267)
cgctagtacgagccgggctaaaatcggccgaggctagccggcagccggatggtcccgcagctcggggccggccatgcttcgcggtccgtggcgccagctttggctctttttcctgctgctgctcccgggcgcgcctgagccccgcggcgcctccaggccgtgggagggaaccgacgagccgggctcggcctgggcctggccgggcttccagcgcctgcaggagcagctcagggcggcgggtgccctctccaagcggtactggacgctcttcagctgccaggtgtggcccgacgactgtgacgaggacgaggaggcagccacggggcccctgggctggcgccttcctctgttgggccagcggtacctggacctcctgaccacgtggtactgcagcttcaaagactgctgccctagaggggattgcagaatctccaacaactttacaggcttagagtgggacctgaatgtgcggctgcatggccagcatttggtccagcagctggtcctaagaacagtgaggggctacttagagacgccccagccagaaaaggcccttgctctgtcgttccacggctggtctggcacaggcaagaacttcgtggcacggatgctggtggagaacctgtatcgggacgggctgatgagtgactgtgtcaggatgttcatcgccacgttccactttcctcaccccaaatatgtggacctgtacaaggagcagctgatgagccagatccgggagacgcagcagctctgccaccagaccctgttcatcttcgatgaagcggagaagctgcacccagggctgctggaggtccttgggccacacttagaacgccgggcccctgagggccacagggctgagtctccatggactatctttctgtttctcagtaatctcaggggcgatataatcaatgaggtggtcctaaagttgctcaaggctggatggtcccgggaagaaattacgatggaacacctggagccccacctccaggcggagattgtggagaccatagacaatggctttggccacagccgtcttgtgaaggaaaacctgattgactacttcatccccttcctgcctttggagtaccgtcacgtgaggctgtgtgcacgggatgccttcctgagccaggagctcctgtataaagaagagacactggatgaaatagcccagatgatggtgtatgtccccaaggaggaacaactcttttcttcccagggctgcaagtctatttcccagaggattaactacatcctgtcatgaaggctagagggaagactt

SEQ ID NO: 2 - protein, 397 aa, Homo sapiens
MLRGPWRQLWLFFLLLLPGAPEPRGASRPWEGTDEPGSAWAWPGFQRLQEQLRAAGALSKRYWTLFSCQVWPDDCDEDEEAATGPLGWRLPLLGQRYLDLLTTWYCSFKDCCPRGDCRISNNFTGLEWDLNVRLHGQHLVQQLVLRTVRGYLETPQPEKALALSFHGWSGTGKNFVARMLVENLYRDGLMSDCVRMFIATFHFPHPKYVDLYKEQLMSQIRETQQLCHQTLFIFDEAEKLHPGLLEVLGPHLERRAPEGHRAESPWTIFLFLSNLRGDIINEVVLKLLKAGWSREEITMEHLEPHLQAEIVETIDNGFGHSRLVKENLIDYFIPFLPLEYRHVRLCARDAFLSQELLYKEETLDEIAQMMVYVPKEEQLFSSQGCKSISQRINYILS

SEQ ID NO: 3 - DNA, 1285 bp, Homo sapiens
  CDS (74)..(1267)
cgctagtacgagccgggctaaaatcggccgaggctagccggcagccggatggtcccgcagctcggggccggccatgcttcgcggtccgtggcgccagctttggctctttctcctgctgctgctcccgggcgcgcctgagccccgcggcgcctccaggccgtgggagggaaccgacgagccgggctcggcctgggcctggccgggcttccagcgcctgcaggagcagctcagggcggcgggtgccctctccaagcggtactggacgctcttcagctgccaggtgtggcccgacgactgtgacgaggacgaggaggcagccacggggcccctgggctggcgccttcctctgttgggccagcggtacctggacctcctgaccacgtggtactgcagcttcaaagactgctgccctagaggggattgcagaatctccaacaactttacaggcttagagtgggacctgaatgtgcggctgcatggccagcatttggtccagcagctggtcctaagaacagtgaggggctacttagagacgccccagccagaaaaggcccttgctctgtcgttccacggctggtctggcacaggcaagaacttcgtggcacggatgctggtggagaacctgtatcgggacgggctgatgagtgactgtgtcaggatgttcatcgccacgttccactttcctcaccccaaatatgtggacctgtacaaggagcagctgatgagccagatccgggagacgcagcagctctgccaccagaccctgttcatcttcgatgaagcggagaagctgcacccagggctgctggaggtccttgggccacacttagaacgccgggcccctgagggccacagggctgagtctccatggactatctttctgtttctcagtaatctcaggggcgatataatcaatgaggtggtcctaaagttgctcaaggctggatggtcccgggaagaaattacgatggaacacctggagccccacctccaggcggagattgtggagaccatagacaatggctttggccacagccgtcttgtgaaggaaaacctgattgactacttcatccccttcctgcctttggagtaccgtcacgtgaggctgtgtgcacgggatgccttcctgagccaggagctcctgtataaagaagagacactggatgaaatagcccagatgatggtgtatgtccccaaggaggaacaactcttttcttcccagggctgcaagtctatttcccagaggattaactacttcctgtcatgaaggctagagggaagactt

SEQ ID NO: 4 - protein, 397 aa, Homo sapiens
MLRGPWRQLWLFLLLLLPGAPEPRGASRPWEGTDEPGSAWAWPGFQRLQEQLRAAGALSKRYWTLFSCQVWPDDCDEDEEAATGPLGWRLPLLGQRYLDLLTTWYCSFKDCCPRGDCRISNNFTGLEWDLNVRLHGQHLVQQLVLRTVRGYLETPQPEKALALSFHGWSGTGKNFVARMLVENLYRDGLMSDCVRMFIATFHFPHPKYVDLYKEQLMSQIRETQQLCHQTLFIFDEAEKLHPGLLEVLGPHLERRAPEGHRAESPWTIFLFLSNLRGDIINEVVLKLLKAGWSREEITMEHLEPHLQAEIVETIDNGFGHSRLVKENLIDYFIPFLPLEYRHVRLCARDAFLSQELLYKEETLDEIAQMMVYVPKEEQLFSSQGCKSISQRINYFLS

SEQ ID NO: 5 - DNA, 2303 bp, Mus musculus
tcatgtttcttggcgctctctggttgctgctgctcctgccgctgcgccccccgggagcgcagggccaggaggccgacgagcccaccccttggccgagcgtcaaaggcctgaaggagcagctgaggacggccggtgccctctccaaacggtactgggagcttttcagctgcaccttgtggcccgatcactgtgaagaccaggagacccccgtgccgcctctgggctggagccttcctctgtggggccggcggtcgctggatgtgctcactgcatggctctgccactttcaggactgctgcagcggcggcggcgattgcaggatctccaacaacttgacaggcttagaatcagacttgtgtgtacgactgcacggccagcatctcgctagcaagctggtcctaagagcagtgaagggctacttagagatgccccaagtaggcaaggccctggctctgtcattccacggctggtctggcacaggcaagaacttcgtggcacggatactgatggacaacctgtatcgggacggcatgaggagtgactgtgtcaagatgtttatttctaccttccactttccacaccccaagtatgtggacacgtacaaggaagagttgcagaggcagatgcaggagacgcagtggcgctgccaccagagcacgttcgtctttgacgaagcggagaagctgcacccggggctgctggagctgcttgaaccctacctggaaccgaggagccctgaggcccgtggagttgaggcgccccgagccatcttcctttttctcagcaacctcggaggcagtgtcatcaatgaggtagtcctgagtttgcttaaggctggatggtccagggaggaaattacgacgcagcacttggaggtgccccttcaggctgagatcatggaggctgcagacagcagctttggctccagcggtctcctgaagaaacaccttattgaccactttatccccttcctgccactggagtaccgccatgtgaggctgtgcgtccgagatgccttcctgggccaggatctcccatacacagaagagaccctggatgaaatcgccaagatgatgacatatgtccctgaggaagagcggcttttctcctctcagggctgcaaatccatttcccagagaatcaacctcttcctgccttgaaagtgactcctgtgcaccctaaacaatcctccacaagagacagccatctgccaggtgccctggcgtccagcaatcactgctttcagctggtgtgcaagaggcagttcctaacaaccacttggtgccttaaaaaacctacattctagacaatctagactgagggtcaggaagccagtggaacggaaggtccaagtaccaaaggatcctcagaacactccctacagtctcctggcaccgtagctcttcctggaaggcagctggagttcatatgagcctgaggctctaggatctttgggctacagaacaaggaatatgtgtggactagaggcgtgtggtccaggtctgcaggtctggctggctttccttgctttccggcaacgtggggagaggggcagcttctgcatgtgagaccagcaagggccttaggctaaaggacaaaccgaagctttcaaaacaggatgggtgtggcctagcactgtagtaggctaatatgggagaagagagctaaagaaacctgagctgcctcgtttgagcagcctgccaggcttgaccaaaacacaggtgtatggatagctaaggcatgttctgttgtgcagactgaaaactttttaaagaggcctcgtcatgtgcaattaaatcttgtatttttctaacatcctttcttactgagacctggtccttgcactgccctggcaggctggcacaagcacgtgctgtctgtcctacgtgctggtctcaggctactttagagcttcttactaaactcctctgactcagccctcgaacacacgtgttctccgctccagctctgacaagtggttcctgaggctgatgtggcgaggcaggtcagtaccacctgtctttagaataccatgggccattcttttctggtcccttcacactttttaaacatacaggtctttgtgttttctcagaatttacccctcttattcaaagtcctgctcagcactctcacctttagttcttgtaaaaatggaccatttctgcaacccccacttaccagtacatgtgtactcactcacctgccttaaggctagggaccacctagcccagccatattcaaataactgaaataaaggagtgtgagtgaggtggtagaggactggccttcagcaggctcag

SEQ ID NO: 6 - protein, 385 aa, Mus musculus
MFLGALWLLLLLPLRPPGAQGQEADEPTPWPSVKGLKEQLRTAGALSKRYWELFSCTLWPDHCEDQETPVPPLGWSLPLWGRRSLDVLTAWLCHFQDCCSGGGDCRISNNLTGLESDLCVRLHGQHLASKLVLRAVKGYLEMPQVGKALALSFHGWSGTGKNFVARILMDNLYRDGMRSDCVKMFISTFHFPHPKYVDTYKEELQRQMQETQWRCHQSTFVFDEAEKLHPGLLELLEPYLEPRSPEARGVEAPRAIFLFLSNLGGSVINEVVLSLLKAGWSREEITTQHLEVPLQAEIMEAADSSFGSSGLLKKHLIDHFIPFLPLEYRHVRLCVRDAFLGQDLPYTEETLDEIAKMMTYVPEEERLFSSQGCKSISQRINLFLP

SEQ ID NO: 7 - DNA, 149 bp, Mus musculus
agctaaagaaacctgagctgcctcgtttgagcagcctgccaggcttgaccaaaacacaggtgtatggatagctaaggcatgttctgttgtgcagactgaaaactttttaaagaggcctcgtcatgtgcaattaaatcttgtatttttct

SEQ ID NO: 8 - DNA, 15 bp, Mus musculus
gagtttcatttcgga

SEQ ID NO: 9 - DNA, 99 bp, Homo sapiens
  CDS (1)..(99)
gcggagattgtggagaccatagacaatggctttggccacagccgtcttgtgaaggaaaacctgattgactacttcatccccttcctgcctttggagtac

SEQ ID NO: 10 - protein, 33 aa, Homo sapiens
AEIVETIDNGFGHSRLVKENLIDYFIPFLPLEY

SEQ ID NO: 11 - DNA, 90 bp, Homo sapiens
  CDS (1)..(90)
gcggagattgtggagaccataggtttttcatttctaacaaccagatggccccacctggacctgccaaccagttctgtggcccccacctag

SEQ ID NO: 12 - protein, 29 aa, Homo sapiens
AEIVETIGFSFLTTRWPHLDLPTSSVAPT

SEQ ID NO: 13 - protein, 316 aa, Homo sapiens
ATGPLGWRLPLLGQRYLDLLTTWYCSFKDCCPRGDCRISNNFTGLEWDLNVRLHGQHLVQQLVLRTVRGYLETPQPEKALALSFHGWSGTGKNFVARMLVENLYRDGLMSDCVRMFIATFHFPHPKYVDLYKEQLMSQIRETQQLCHQTLFIFDEAEKLHPGLLEVLGPHLERRAPEGHRAESPWTIFLFLSNLRGDIINEVVLKLLKAGWSREEITMEHLEPHLQAEIVETIDNGFGHSRLVKENLIDYFIPFLPLEYRHVRLCARDAFLSQELLYKEETLDEIAQMMVYVPKEEQLFSSQGCKSISQRINYFLS

SEQ ID NO: 14 - protein, 312 aa, Homo sapiens
AVEPISLGLALAGVLTGYIYPRLYCLFAECCGQKRSLSREALQKDLDDNLFGQHLAKKIILNAVFGFINNPKPKKPLTLSLHGWTGTGKNFVSKIIAENIYEGGLNSDYVHLFVATLHFPHASNITLYKDQLQLWIRGNVSACARSIFIFDEMDKMHAGLIDAIKPFLDYYDLVDGVSYQKAMFIFLSNAGAERITDVALDFWRSGKQREDIKLKDIEHALSVSVFNNKNSGFWHSSLIDRNLIDYFVPFLPLEYKHLKMCIRVEMQSRGYEIDEDIVSRVAEEMTFFPKEERVFSDKGCKTVFTKLDYYYD

SEQ ID NO: 15 - protein, 313 aa, Mus musculus
AVEPISLSLALAGVLTTYISYPRLYCLFAECCGQMRSLSREALQKDLDNKLFGQHLAKKVILNAVSGFLSNPKPKKPLTLSLHGWTGTGKNFASKIIAENIYEGGLNSDYVHLFVATLHFPHASNITQYKDQLQMWIRGNVSACARSIFIFDEMDKMHAGLIDAIKPFLDYYDVVDEVSYQKAIFIFLSNAGAERITDVALDFWKSGKQREEIKLRDMEPALAVSVFNNKNSGFWHSSLIDRNLIDYFVPFLPLEYKHLKMCIRVEMQSRGYEVDEDIISKVAEEMTFFPKEEKVFSDKGCKTVFTKLDYYLD

SEQ ID NO: 16 - protein, 266 aa, Homo sapiens
DLEEKLFGQHLATEVIFKALTGFRNNKNPKKPLTLSLHGWAGTGKNFVSQIVAENLHPKGLKSNFVHLFVSTLHFPHEQKIKLYQDQLQKWIRGNVSACANSVFIFDEMDKLHPGIIDAIKPFLDYYEQVDGVSYRKAIFIFLSNAGGDLITKTALDFWRAGRKREDIQLKDLEPVLSVGVFNNKHSGLWHSGLIDKNLIDYFIPFLPLEYRHVKMCVRAEMRARGSAIDEDIVTRVAEEMTFFPRDEKIYSDKGCKTVQSRLDFH

SEQ ID NO: 17 - protein, 298 aa, Mus musculus
ALTGYLSYTDFYCRFTECCHEERPLNTSALKLDLEEKLFGQHLATEVILKALTGFRNNKNSKKPLTLSLHGWAGTGKNFISQIVAENLYPKGLKSNFVHLFVSTLHFPHEQKIKVYQDQLQKWIRGNVSACGSSVFIFDEMDKLHPGIIDAIKPFLDYYEQVDGISYRRAIFIFLSNAGGDLITKTALDFWRAGRKREEDQLKDMEHVLSVGVFNNKHSGLWHSGLIDKNLIDYFIPFLPLEYKHVKMCVRAEMRARGAAVDEDVVTSVADEMTFFPKDEKIYSDKGCKTVQSRLDFH

SEQ ID NO: 18 - protein, 294 aa, Drosophila melanogaster
YCRFAECCDDRNIPARIDELERSLERTLIGQHIVRQHIVPALKAHIASGNKSRKPLVISFHGQPGTGKNFVAEQIADAMYLKGSRSNYVTKYLGQADFPKESEVSNYRVKINNAVRDTLRSCPRSLFIFDEVDKMPSGVFDQLTSLVDYNAFVDGTDNTKAIFIFLSNTAGSHIASHLGSVMKNGRLREDTRLSDFEPLLRKAAYNMDGGMKKTTMIESHVIDHFIPFLPMEKAHVIKCLEAELLRWRRDPKQANNQKIIEDIINSSISYDRTHSLFAISGCKTLEKKVAMAIY

SEQ ID NO: 19 - protein, 290 aa, Caenorhabditas elegans
CLFYTCCGETDIFNYHALYKDFDNKIFGQHLMAESVVHSIKSHWHNEHSQKPLVLSFHGGTGTGKNYVTEIIVNNTYRSGMHSPFVNYFVATNNFPNKKYIEDYKLELKDQLIRSARRCQRSIFIFDETDKLQSELIQVIKPFLDYYPAVFGVDFRKTIFIFLSNKGSKEIANIALEHHENGKIRSQLELKHFERTLMLSAFNEEGGLRNTDMISNQLIDHFIPFLPLSKFYVSQCIQVHLRKRGRHDLAKDGEFMQRVLDSLEFFPESSKIFSSSGCKRVNAKTDLEIS

SEQ ID NO: 20 - protein, 291 aa, Caenorhabditas elegans
CYLYECCHEPDVNFNYHTLDADIANLLFGQHLVKDVVVNSIKSHWYNENPRKPLVLSFHGYTGSGKNYVAEIIANNTFRLGLRSTFVQHIVATNDFPDKNKLEEYQVELRNRILTTVQKCQRSIFIFDEADKLPEQLLGAIKPFLDYYSTISGVDFRRSIFILLSNKGGGEIARITKEQYESGYPREQLRLEAFERELMNFSYNEKGGLQMSELISNHLIDHFVPFLPLQREHVRSCVGAYLRKRGRGDLVSNVDFVERVLNSLQYFPESSKAFSSSGCKRVDAKTDLEMA

SEQ ID NO: 21 - protein, 281 aa, Caenorhabditas elegans
CSTENGLELHRDLSRFIYGQHLVIDTVVRSIKSHWHNEHPQKPLVLSFHGGPGTGKNYVTEIIAKNTFRSGLQSPYVKYFVATKDFPNNKHIEDYKLKLKEQLIQSADGCDRSIFVFDEVDKLQSELVQTIKPFLDFYPAVFEVDFRKTTFIFLSNKGSSEIANIALEHRRNLKKRSQLELKHFERTLMSHAFNEKGGLRNTELISNQLIDHYIPFLPLSKFYVSQCIQVHLRKRGRHDLAKDGEFMQRVLDSLEFFPESSKVFSSSGCKRVDAKTELEIS

SEQ ID NO: 22 - protein, 224 aa, Homo sapiens
GLEWDLNVRLHGQHLVQQLVLRTVRGYLETPQPEKALALSFHGWSGTGKNFVARMLVENLYRDGLMSDCVRMFIATFHFPHPKYVDLYKEQLMSQIRETQQLCHQTLFIFDEAEKLHPGLLEVLGPHLERRAPEGHRAESPWTIFLFLSNLRGDIINEVVLKLLKAGWSREEITMEHLEPHLQAEIVETIDNGFGHSRLVKENLIDYFIPFLPLEYRHVRLCAR

SEQ ID NO: 23 - protein, 216 aa, Triticum aestivum
GLADRLHQRVVGQYEAVNAVGEAVLRSRAGLGRPQQPTGSFLFLGPTGVGKTELAKALAEQLFDDENLLVRIDMSEYMEQHSVARLIGAPPGYVGHEEGGQLTEQVRRRPYSVILFDEVEKAHVAVFNTLLQVLDDGRLTDGQGRTVDFRNTVIIMTSNLGAEHLLAGMVGNSMKVARDLVMQEVRRHFRPELLNRLDEIVIFDPLSHEQLRKVAR

SEQ ID NO: 24 - protein, 218 aa, Mus musculus
PLEQRLKEHIIGQESAIATVGAAIRRKENGWYDEEHPLVFLFLGSSGIGKTELAKQTAKYMHKDAKKGFIRLDMSEFQERHEVAKFIGSPPGYIGHEEGGQLTKKLKQCPNAVVLFDEVDKAHPDVLTIMLQLFDEGRLTDGKGKTIDCKDAIFIMTSNVASDEIAQHALQLRQEALEMSRNRIAENLGDVQMSDKITISKNFKENVIRPILKAHFRR